(19) United States
(12) Patent Application Publication
(10) Pub. No.: US 2003/0129638 A1
(43) Pub. Date: Jul. 10, 2003

(54) Reagents for antagonizing the protein-protein interaction between Raf-1 and apoptosis signal-regulating kinase and uses therefor

(76) Inventors: Haian Fu, Atlanta, GA (US); Jing Chen, Brookline, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/314,568

(22) Filed: Dec. 9, 2002

Related U.S. Application Data
(60) Provisional application No. 60/340,399, filed on Dec. 7, 2001.

(51) Int. Cl. C12Q 1/68; G01N 33/574; A61K 39/395; C12N 9/12; A61K 48/00; C07K 16/40

(52) U.S. Cl. 435/6; 435/7.23; 514/44; 435/194; 424/146.1; 530/388.26

(57) ABSTRACT

The present invention identifies a novel protein-protein interaction between Raf-1 and apoptosis signal-regulating kinase 1, thereby identifying a molecular basis for cross-talk between the Raf-1-mediated signaling and ASK-1-meditated apoptotic signaling. The invention provides methods for screening for agents that are capable of disrupting the disclosed protein-protein interaction or which are capable of modulating ASK1-mediated apoptosis. The invention further provides Raf-1 binding polypeptides, derived from the N-terminal regulatory domain of ASK1, that find utility as therapeutic agents, as reagents for establishing screening assays, as an immunogens to elicit peptide specific antibodies and as paradigmatic agents for the design or identification of small molecules that share a sufficiently similar structure so as to inhibit or promote the disclosed ASK1/Raf-1 interaction.

### CROSS-REFERENCE TO RELATED APPLICATIONS

This application claims the benefit of U.S. Provisional Application Serial No. 60/340,399, filed Dec. 7, 2001, the contents of which are herein incorporated by reference.

### FEDERALLY SPONSORED RESEARCH OR DEVELOPMENT

The research underlying this invention was supported in part with funds from National Institutes of Health/National Institute of General Medical Sciences (NIH/NIGMS) grants GM53165, GM60033, and American Heart Association grant number 9950226N. The United States Government may have an interest in the subject matter of the invention.

### FIELD OF THE INVENTION

The present invention relates generally to cell biology and to the molecular mechanisms underlying the coordination, integration, and balance of proapoptotic and antiapoptotic signaling cascades that regulate diverse cellular processes, including differentiation, proliferation, survival, or apoptosis.

### BACKGROUND OF THE INVENTION

Apoptosis is a highly regulated physiological process of cell death that plays a critical role in normal development, as well as in the pathophysiology of a variety of diseases. Cells are continuously exposed to conflicting extracellular signals capable of mediating “death” (e.g., proapoptotic) and “survival” (e.g., antiapoptotic). Disruption of the coordination and/or balance of the molecular mechanisms responsible for regulating these signals is known to be associated with the pathogenesis of a wide array of diseases, including neurodegeneration, autoimmune diseases, cancer, heart disease, and other disorders (Jacobson et al. (1997) Cell 88:347-354 and Rudin and Thompson (1997) Annu. Rev. Med. 48:267-281).

Extensive research in recent years has identified protein kinases, and their associated proteins, as key molecules participating in a conserved intracellular signaling pathway that mediates the highly ordered process of apoptotic cell death (for review, see Ellis et al. (1991) Annu. Rev. Cell Biol. 7:663-698; Cryns and Yuan (1998) Genes Dev. 12:1551-1570; and Ashkenazi and Dixit (1998) Science 281:1305-1308). The mitogen-activated protein kinase (MAPK) cascade is evolutionary well conserved in all eukaryotic cells. Generally speaking, the MAPK cascade typically comprises three kinases that participate in a sequential activation pathway comprising a MAP kinase kinase kinase (MAPKKK), a MAP kinase kinase (MAPKK), and a MAP kinase (MAPK)(Matsuzawa and Ichijo (2001) J. Biol. Chem. 130:1-8).

Homeostasis in mammalian cells is known to be dependent upon the continuous regulation and integration of death and survival signals from the extracellular environment. In fact, many of the molecules responsible for both apoptotic and survival signals are subject to autoregulation, for example by negative regulation of their own signals. It has also become clear that apoptotic pathways are intimately associated with cell survival pathways to ensure that cell death occurs only when needed.

This association or cross-talk between distinct pathways is partly achieved by specific targeting of the key elements of proapoptotic signaling cascades by antiapoptotic mechanisms. While many of the elements of the regulatory networks controlling apoptosis have been determined, the molecular mechanism of action and the patterns of interaction of these elements remain controversial. Therefore, it is apparent that there is a continuing need for methods and reagents that will facilitate the elucidation of the molecular mechanisms and signal transduction pathways that mammalian cells utilize to integrate diverse extracellular signals. In particular there is a need for a greater understanding of the regulatory mechanisms that control cell fate, such as survival, proliferation, differentiation, or apoptosis.

### SUMMARY OF THE INVENTION

The invention described herein is based on the discovery of a MEK-ERK-independent prosurvival function of Raf-1 that can be attributed to a protein-protein interaction between Raf-1 and apoptosis signal-regulating kinase 1 (ASK1). This interaction provides a molecular basis for cross-talk between the Raf-1 signaling pathway and ASK-1-mediated apoptosis. The present invention identifies a novel target for therapeutic intervention, and provides methods and reagents for modulating ASK1-dependent signaling and for screening for agents that are capable of disrupting the disclosed protein-protein interaction between Raf-1 and ASK1.

Discovery of the disclosed Raf-1/ASK1 interaction provides a novel target for therapeutic agents that may be useful to treat diseases and disorders associated with aberrant Raf-1 and/or ASK1 expression. These diseases include various types of cancers that may result from the dysregulation of Raf-1 and/or ASK1 expression or from an aberrant interaction between Raf-1 and ASK1. Other diseases include inflammatory diseases, such as rheumatoid arthritis, that may involve ASK1 signaling. This aspect of the invention is premised on the assumption that suppression of ASK1 may provide a general mechanism for promoting cell survival.

The ability to modulate ASK-1 dependent signaling affords an opportunity to regulate the signals underlying the coordination and balance between proapoptotic and antiapoptotic signaling cascades, thereby providing a means to modulate diverse cellular processes including differentiation, proliferation, survival, or apoptosis. Such modulation of ASK1 activity can provide a means to inhibit neoplastic conditions and a means to modulate inflammatory processes.

The present invention also provides methods and reagents for screening for agents that are modulators of ASK1 and/or MEK-ERK independent Raf-1 function. In one embodiment, the invention provides a method for modulating ASK1-mediated apoptosis of a cell which comprises administering to the cell an agent which is capable of inhibiting or promoting the disclosed protein-protein interaction between Raf-1 and ASK1.

In an alternative embodiment, the invention provides a method of screening for polypeptides that modulate human ASK1-mediated cell death comprising incubating cells comprising human ASK1, a human Raf-1 binding target, and a heterologous nucleic acid sequence encoding a fusion peptide which comprises a candidate modulatory polypeptide under conditions, whereby but for the presence of the heterologous gene product ASK1 would bind the Raf-1 binding target; exposing the cells to an extracellular signal sufficient to trigger ASK1-mediated apoptosis, and measuring the percentage of cell death that occurs in the cell population. A difference between the percentage of death that occurs in the presence of the fusion peptide relative to the percentage of death that occurs in the absence of the fusion peptide indicates that the polypeptide modulates human ASK1-mediated apoptosis.

The above-described method can be modified to identify polypeptides that modulate cell death in a MEK-ERK independent manner by using a Raf-1 binding target that is catalytically inactive. The disclosed method can also be designed to identify polypeptides that modulate the effects of triggering particular ASK1-dependent signaling cascades. For example, the extracellular signal used to trigger apoptosis can be a signal such as exposure to a cytokine such as TNF, treatment with a chemotherapeutic drug (e.g., cisplatin and paclitaxel), or exposure to oxidative stress. Thus, in one embodiment, the invention provides a method of inhibiting TNFα-mediated apoptosis in a cell.

The invention further provides methods and reagents for screening for agents that disrupt the disclosed protein-protein interaction between Raf-1 and ASK1. In one embodiment, the invention provides a set of Raf-1 binding peptides derived from the N-terminal regulatory domain of ASK1 that are demonstrated herein to contain polypeptides (e.g., peptides) that inhibit the disclosed binding interaction. The disclosed peptides provide agents capable of modulating the interaction of Raf-1/ASK1 and thereby represent regulators of ASK1-mediated apoptosis.

The Raf-1 binding peptides disclosed herein also provide lead molecules for the development of other agents (e.g., peptidomimetics, peptoids, and small molecule inhibitors) that are capable of regulating ASK1-dependent apoptotic signaling cascades. It is recognized that any one of the polypeptides set forth in SEQ ID NOS:3, 5, 7, 8, 10 and 11 may be used as a paradigm polypeptide for designing and identifying a peptidomimetic that shares sufficient structural similarity to disrupt the binding interaction of Raf-1 and ASK1 and to thereby antagonize ASK1-mediated apoptosis. In addition the peptides provide a valuable reagent for use as controls in establishing screening assays designed to identify and/or characterize other agents capable of regulating ASK1-mediated apoptosis.

The ASK1 peptides disclosed herein are also useful as immunogens and can be used to elicit antibodies (e.g., monoclonals and polyclonals) that specifically recognize the epitopes comprised within their sequences. Antibodies elicited in response to immunization with the disclosed peptides are a feature of the invention, as is any other polypeptide or agent that is identified as a consequence of its immunoreactivity with an antibody produced using one of the disclosed peptides of the invention as an immunogen.

### BRIEF DESCRIPTION OF DRAWINGS

FIG. 1A provides a set of photomicrographs illustrating the results of a transient transfection experiment that was performed to evaluate the effects of Raf-1 on ASK1-mediated apoptosis. Apoptotic cell death was monitored by nuclear morphology. The fraction of transfected cells with fragmented nuclei was quantified in a blind manner.

FIG. 1B is a graphic summary (upper panel) of the fraction of transfected cells exhibiting apoptotic nuclear morphology. The lower panel of FIG. 1B is a graphic representation of Western blot results that were obtained using cell lysates from each of the transiently transfected samples.

FIG. 1C is a histogram summarizing the results of a DNA content-based flow cytometry assay performed to analyze transfected COS7 cells. Transfected cells (eGFP-positive) were placed in various phases of the cell cycle based on their DNA content. Apoptotic cells with fragmented DNA (subG₀) are indicated.

FIG. 1D is a graphic summary showing the amount of apoptosis caused by expression of the transfected plasmids.

FIG. 2A is a set of photomicrographs obtained using a morphology-based apoptosis assay performed to evaluate the effects of Raf-1 on ASK1-induced apoptosis. Shrunken apoptotic cells with rounded-up shape were scored as apoptotic (arrows).

FIG. 2B is a graphic summary of the effect of MEK inhibition by PD98059 on Raf-1 function. Six hours after transfection as in A, HeLa cells were treated with PD98059 (60 μM) or vehicle (DMSO) for 18 h before being stained with 5-bromo-4-chloro-3-indolyl-D-galactopyranoside and scored for apoptosis in a blind fashion. Specific apoptosis is derived by subtracting the level of apoptosis seen in pcDNA3-transfected cells. At least 500 cells were scored for each sample. Results shown are representative of three independent experiments.

FIG. 2C shows a Western blot performed to evaluate the effects of PD98059 inhibition on ERK1/2 activation. Cell lysates from HeLa cells treated with 60 μM PD98059 or vehicle were probed by Western blotting with either an ERK1/2 activation-specific antibody (Upper; Cell Signaling Technology) or pan-ERK1/2 antibody (Lower; Cell Signaling Technology).

FIG. 2D is a graphic summary of the effect of MEK1 overexpression on ASK1-induced apoptosis. HeLa cells were transfected with a β-gal reporter together with indicated expression vectors for 12 h, serum-starved for 24 h, and scored for apoptosis as in A. Data are summary of three independent experiments.

FIG. 2E is a graphic summary of the effect of MEK1 expression on ERK1/2 activation. Lysates from HeLa cells treated as in D were subjected to SDS/PAGE and Western blotting with ERK1/2 activation-specific or pan antibodies or anti-MEK antibody (Santa Cruz Biotechnology).

FIG. 3A is a Western blot of HA-ASK1 immunocomplexes. COS7 cells were transfected with the expression vector for HA-ASK1 or HA-CAB1 together with FLAG-Raf-1 (Zhang (1999) Proc. Natl. Acad. Sci. USA 96:8511-8515). After 48 h, cell lysates were prepared, and ASK1 or CAB1 was immunoprecipitated with anti-HA antibody. Western blots were developed with antibodies to Raf-1 and HA (Upper). The Western blot in the lower panel shows the expression levels of Raf-1 and HA-ASK1 or HA-CAB1 in the lysates.

FIG. 3B is a Western blot of Raf-1 immunocomplexes. Polyclonal anti-Raf-1 antibody (Santa Cruz Biotechnology) was used to immunoprecipitate Raf-1. HA-ASK1 was detected in Raf-1 immunoprecipitates with anti-HA antibody.

FIG. 3C is a Western blot of immunocomplexes of endogenous Raf-1 and ASK1 in L929 cells. Immunoprecipitates were prepared from 929 cell lysates (left lane) using either anti-Raf-1 monoclonal antibody (Transduction Laboratories) or anti-HA antibody as a negative control and probed for ASK1 using the antibody DAV (Saitoh et al. (1998) EMBO J. 17:2596-2606; Upper). Coimmunoprecipitated ASK1 and Raf-1 comigrate, respectively, with overexpressed HA-ASK1 and FLAG-Raf-1 (Marker lane). Antibody light chains (LC) in the immunoprecipitates are indicated.

FIG. 3D is a Western blot demonstrating that 14-3-3 binding and Raf-1 kinase activity are not required for the Raf-1-ASK1 interaction. COS7 cells were cotransfected with plasmids encoding FLAG-Raf-1WT, catalytically inactive FLAG-Raf^{K375M} (301), or 14-3-3 binding defective FLAG-Raf^{S259/621A} (2SA) and HA-ASK1WT or 14-3-3 binding defective HA-ASK^{S967A} (SA). FLAG-Raf-1 complex was precipitated by using anti-FLAG antibody (Sigma) and probed with anti-ASK1 antibody (Santa Cruz Biotechnology). Expression levels of HA-ASK1 were verified by Western blotting (Lower).

FIG. 4 provides a graphic summary of a HeLa cell morphology-based apoptosis assay performed to determine whether kinase-defective Raf-1 mutants Raf-1^{K375M} and Raf-1^{S259/621A} were capable of binding ASK1.

FIG. 5A is a schematic diagram of ASK1 proteins and mutants used herein to elucidate the region of ASK1 that interacts with Raf-1. The shaded portion of the boxes represents the ASK1 kinase domain. Association of ASK1 mutants with Raf-1 is summarized.

FIG. 5B provides the results of a Western blot. FLAG-Raf-1 was transiently transfected into COS7 cells with HA-ASK1WT or truncated mutants. HA-ASK1 protein complexes were immunoprecipitated and subjected to SDS/PAGE and Western blotting with anti-HA (Middle) and anti-Raf-1 antibodies (Top panel). Lysates from each sample were probed with anti-Raf-1 antibodies (Bottom panel).

FIG. 5C is a Western blot analysis establishing that Raf-1 does not interact with ASK1-ΔN. Raf-1 protein complexes were immunoprecipitated from each sample with anti-Raf-1 antibody and subjected to SDS/PAGE and Western blotting with anti-ASK1 antibody. Overexposure shows the interaction of endogenous Raf-1 with overexpressed HA-ASK1, but even overexpressed Raf-1 was incapable of binding to ASK1-ΔN.

FIG. 5D is a graphic representation of apoptoisis data obtained in a nuclear morphology-based assay using ASK1 mutants to evaluate the effect of the ΔN mutation on ASK1-dependent apoptosis as determined by the nuclear morphology-based apoptotic assay described in FIG. 1. HeLa cells were transfected with plasmids as indicated together with an eGFP marker vector.

FIG. 6A provides a schematic representation of the ASK1 N-terminal domain truncation proteins used herein. Association of ASK1 mutants with Raf-1 is summarized. Positive interaction is represented by (+).

FIG. 6B provides a schematic representation of the ASK1 truncation proteins that inhibit the interaction of ASK1 and Raf-1 and include the amino acid sequence (SEQ ID NO:11) that is common to all of the ASK1 peptides capable of blocking the Raf-1 interaction.

FIG. 7 is a Western blot analysis of Raf-1 immunoprecipitates obtained from transfectants comprising N-terminal domain truncation peptides.

FIG. 8 provides a graphic representation of data obtained from a DNA content-based flow cytometric apoptosis assay. The fraction of transfected cells with sub-G₀ DNA content in each sample was quantified. The data presented are representative of three independent experiments.

FIG. 9 provides the amino acid sequence of full-length wild-type human ASK1.

FIG. 10 provides the nucleotide sequence of human ASK1.

### DETAILED DESCRIPTION OF THE INVENTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

The terms “native protein” and “full-length protein” as used herein refer to a polypeptide corresponding to the deduced amino acid sequence of a human Raf-1 or ASK cDNA or corresponding to the deduced amino acid sequence of a cognate full-length Raf-1 or ASK1 cDNA from a nonhuman mammal.

The term “naturally occurring” or “wild-type” as used herein as applied to a polypeptide or polynucleotide sequence refers to the fact that a sequence can be found in nature. For example, a polypeptide or polynucleotide sequence that is present in an organism (including viruses) that can be isolated from a source in nature and which has not been intentionally modified by man in the laboratory is naturally occurring. Generally, the term naturally occurring refers to an object as present in a non-pathological (undiseased) individual, such as would be typical for the species.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence (e.g., a human ASK1 cDNA sequence). Fragments typically are at least 10 amino acids long, preferably at least 12 amino acids long, usually at least 20 amino acids long or longer, and span the portion of the polypeptide required for intermolecular binding of Raf-1 to ASK1.

The term “analog” as used herein refers to polypeptides which are comprised of a segment of at least 20 amino acids that has substantial identity to a portion of the deduced amino acid sequence of human ASK1 cDNAs, and which has the property of binding to Raf-1 protein, to form a detectable ASK1: Raf-1 complex.

The term “polypeptide” is used herein as a generic term to refer to native protein, fragments, or analogs of Raf-1 or ASK1. Hence, native Raf-1, fragments of Raf-1, and analogs of Raf-1 are species of the Raf-1 polypeptide genus.

As used herein, the twenty conventional amino acids and their abbreviations follow conventional usage (Golub and Gren, eds. (1991) Immunology-A Synthesis (2d ed., Sinauer Associates, Sunderland, Mass.), which is incorporated herein by reference). Stereoisomers (e.g., D-amino acids) of the twenty conventional amino acids, unnatural amino acids such as α, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ξ-N,N,N-trimethyllysine, ξ-N-acetyllysine, O-phosphoserine, N-acetylserine, N-formylmethionine, 3-methylhistidine, 5-hydroxylysine, omega.-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention. Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

Conservative amino acid substitution is a substitution of an amino acid by a replacement amino acid, which has similar characteristics (e.g., those with acidic properties: Asp and Glu). A conservative (or synonymous) amino acid substitution should not substantially change the structural characteristics of the parent sequence (e.g., a replacement amino acid should not tend to break a helix that occurs in the parent sequence, or disrupt other types of secondary structure that characterizes the parent sequence). For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Examples of art-recognized polypeptide secondary and tertiary structures are described in Creighton, ed. (1984) Proteins, Structures and Molecular Principles, Introduction to Protein Structure (1991), W. H. Freeman and Company, New York; Branden and Tooze, Garland Publishing, New York, N.Y.; and Thornton et al. (1991) Nature 354:105, which are incorporated herein by reference.

The term “agent” is used herein to denote a chemical compound (e.g., a polypeptide, a peptidomimetic, a small synthetic or organic molecule), a mixture of chemical compounds, a biological macromolecule, or an extract made from biological materials such as bacteria, plants, fungi, a natural products library or animal (particularly mammalian) cells or tissues. Agents are evaluated for potential activity as modulators and/or inhibitors of ASK1 function by inclusion in screening assays as described below.

The term “ASK1 antagonist” is used herein to refer to agents which inhibit ASK1-mediated cellular processes. ASK1 antagonists typically will inhibit apoptosis. In a particular embodiment the Raf-1 binding peptides derived from the N-terminal regulatory regions of ASK1 disclosed herein antagonize ASK1-mediated apoptosis through a MEK-ERK independent mechanism attributed to a protein-protein interaction between Raf-1 and ASK1. In contradistinction, an ASK1 agonist would promote a particular molecular interaction or cellular function.

The term “protein interaction inhibitor” is used herein to refer to an agent which is identified by one or more screening method(s) of the invention as an agent which selectively inhibits protein-protein binding between a first interacting polypeptide and a second interacting polypeptide. Some protein interaction inhibitors may have therapeutic potential as drugs for human use and/or may serve as commercial reagents for laboratory research or bioprocess control. Protein interaction inhibitors which are candidate drugs may be tested further for activity in assays which are routinely used to predict suitability for use as human and veterinary drugs, including in vivo administration to non-human animals and often including administration to human in approved clinical trials.

As used herein, the term “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), biotinyl groups, predetermined polypeptide epitopes recognized by a secondary reporter (e.g., leucine zipper pair sequences, binding sites for secondary antibodies, metal binding domains, epitope tags). In some embodiments labels are attached by spacer arms of various lengths to reduce potential steric hindrance.

As used herein, “substantially pure” means an object species is the predominant species present (i.e., on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

Apoptosis Signal-Regulating Kinase 1 (ASK1)

Apoptosis signal-regulating kinase 1 (ASK1), a Ser/Thr kinase, is a mitogen-activated protein MAPKKK that activates both the MKK4-JNK and MKK3/6-p38 signaling cascades. ASK1 is a proapoptotic kinase that is a pivotal component of cytokine and stress-induced cell death (Wang et al. (1996) J. Biol. Chem. 271:31607-31611; Ichijo et al. (1997) Science 275:90-94; Saitoh et al. (1998) EMBO J. 17:2596-2606; Wang et al. (1998) J. Biol. Chem. 273:4928-4936 and Chang et al. (1998) Science 281:1860-1863). Due to its central role in mediating apoptotic signaling, ASK1 is highly regulated by multiple molecular mechanisms.

ASK1 is an important mediator of apoptotic signaling initiated by a variety of death stimuli, including tumor necrosis factor, Fas activation, oxidative stress, and DNA damage (Ichijo et al. (1997) Science 275:90-94; Chang et al. (1998) Science 281:1860-1863; Saitoh et al. (1998) EMBO J. 17:2596-2606; Gotoh and Cooper (1998) J. Biol. Chem. 273:17477-17482 and Chen et al. (1999) Oncogene 18:173-180). ASK1 appears to be a general mediator of cell death because it is responsive to numerous stress signals, including oxidative stress (Saitoh et al. (1998) EMBO J. 17:2596-2606; Gotoh and Cooper (1998) J. Biol. Chem. 273:17477-17482). Overexpression of ASK1 has been demonstrated to be sufficient to induce apoptosis in many cell types (Ichijo et al. (1997) Science 275:90-94 and Chang et al. (1998) Science 281:1860-1863). For example, the kinase activity of ASK1 is stimulated by tumor necrosis factor (TNF) via members of the TNF receptor-associated factor (TRAF) family (Ichijo et al. (1997) Science 275:90-94; Saitoh et al. (1998) EMBO J. 17:2596-2606; Wang et al. (1998) J. Biol. Chem. 273:4928-4936; Chang et al. (1998) Science 281:1860-1863; and Nishitoh et al. (1998) Mol. Cell. 2:389-395) by Fas ligation via the Daxx protein (Chang et al. (1998) Science 281:1860-1863), by UV radiation, and by exposure to DNA-damaging agents such as cisplatin and paclitaxel (Wang et al. (1998) J. Biol. Chem. 273:4928-4936, Chen et al. (1999) Oncogene 18:173-180).

Consistent with its role in apoptotic signaling, dominant negative mutants of ASK1 can inhibit tumor necrosis factor α and Fas ligation-induced cell death (Ichijo et al. (1997) Science 275:90-94; and Chang et al. (1998) Science 281:1860-1863), and overexpression of ASK1 is sufficient to cause apoptosis in a number of cell lines through a mitochondria-dependent caspase activation pathway (Hatai et al. (2000) J. Biol. Chem. 275:26576-2658). Thus, suppression of ASK1 may provide a general mechanism for cell survival. Indeed, multiple mechanisms have been described that directly control ASK1 function. For example, the binding of reduced thioredoxin has been shown to inhibit ASK1-induced apoptosis, which may couple intracellular redox state to the regulation of ASK1 activity (Saitoh et al. (1998) EMBO J. 17:2596-2606 and Gotoh and Cooper (1998) J. Biol. Chem. 273:17477-17482). The phosphoserine-binding protein 14-3-3 can inhibit the proapoptotic function of ASK1 through binding to Ser-967 of ASK1, which is phosphorylated by an unknown survival signaling kinase (Zhang et al. (1999) Proc. Natl. Acad. Sci. USA 96:8511-8515).

Raf-1

The Ser/Thr kinase Raf-1 is a protooncogene product that is a central component in many prosurvival mechanisms involved in normal cell growth and oncogenic transformation. Thus, in contrast to the proapoptotic signals originating from ASK1, Raf-1 activation utilizes a MEK-ERK-dependent mechanism to mediate a signaling cascade that functions to provide cells with survival, and which plays a role in diverse cellular processes such as proliferation, differentiation, and transformation.

Upon activation, Raf-1 phosphorylates mitogen-activated protein kinase (MEK), which in turn activates mitogen-activated protein kinase/extracellular signal-regulated kinases (MAPK/ERKs), leading to the propagation of signals. Depending on specific stimuli and cellular environment, the Raf-1-MEK-ERK cascade regulates diverse cellular processes. Recently, Raf-1 activation of the MEK-ERK pathway has been associated with inhibition of apoptosis, leading to cell survival (Cleveland et al. (1994) Oncogene 9:2217-2226; Xia et al. (1995) Science 270:1326-1331; Erhardt et al. (1999) Mol. Cell. Biol. 19:5308-5315; and Le Gall et al. (2000) Mol. Biol. Cell 11:1103-1112).

Diverse signaling pathways, such as those mediated by tyrosine kinase receptors and heterotrimeric G protein-coupled receptors, converge on Raf-1 through Ras and other mechanisms (Morrison and Cutler (1997) Curr. Opin. Cell Biol. 9:174-179; Kolch (2000) Biochem. J. 351:289-305). Raf-1 activation initiates a mitogen-activated protein kinase (MAPK) cascade that comprises a sequential phosphorylation of the dual-specific MAPK kinases (MEKs) and the extracellular signal-regulated kinases (ERKs). In turn, the Raf-1-MEK-ERK cascade regulates diverse cellular processes such as proliferation and differentiation.

Consistent with a critical role of the MEK-ERK pathway in antiapoptotic signaling pathways, the treatment of cells with either MEK inhibitors or dominant inhibitory MEKs has been reported to inhibit the antiapoptotic function of Raf (Erhardt et al. (1999) Mol. Cell. Biol. 19:5308-5315 and Le Gall et al. (2000) Mol. Biol. Cell 11:1103-1112.

The prosurvival function of the MEK-ERK pathway appears to be mediated by dual mechanisms. A transcription-dependent mechanism involves the activation of cAMP response element-binding protein by ribosomal S6 kinases, leading to increased transcription of prosurvival genes, whereas a transcription-independent mechanism allows phosphorylation of proapoptotic proteins such as Bad, leading to its inactivation (Bonni et al. (1999) Science 286:1358-1362; Scheid et al. (1999) J. Biol. Chem. 274:31108-31113 and Shimamura et al. (2000) Curr. Biol. 10:127-135). In support of this model, genetic analysis in Drosophila demonstrated that activated ERK pathway inhibits the expression and activity of the proapoptotic protein Hid (Kurada and White (1998) Cell 95:319-329 and Bergmann et al. (1998) Cell 95:331-341). Thus, the Raf-activated MEK-ERK pathway may promote cell survival by targeting proteins critical for mediating apoptosis.

The invention disclosed herein describes a physical and functional interaction of Raf-1 with ASK1, suggesting a novel prosurvival mechanism for Raf-1 independent of the MEK-ERK pathway. The disclosed prosurvival effect of Raf-1, is mediated by catalytically inactive forms and wild-type forms of Raf-1, which suggests that the prosurvival function disclosed herein represents a kinase-independent function of Raf-1. Thus, Raf-1 may promote cell survival through its protein-protein interactions in addition to its established MEK kinase function.

Production of ASK1 Fusion Polypeptides

The polypeptide sequences set forth in SEQ ID NOS:2-11 may be synthesized by chemical methods or produced by in vitro translation systems using a polynucleotide template to direct translation. Methods for expression of heterologous proteins in recombinant hosts, chemical synthesis of polypeptides, and in vitro translation are well known in the art and are described further in Maniatis et al. (1989) Molecular Cloning: A Laboratory Manual (2d, Cold Spring Harbor, N.Y.); Berger and Kimmel (1987) Methods in Enzymology:Guide to Molecular Cloning Techniques, Vol. 152 (Academic Press, Inc., San Diego, Calif.); Merrifield (1969) J. Am. Chem. Soc. 91:501; Chaiken (1981) CRC Crit. Rev. Biochem. 11:255; Kaiser et al. (1989) Science 243:187; Merrifield (1986) Science 232:342; Kent (1988) Ann. Rev. Biochem. 57:957; and Offord (1980) Semisynthetic Proteins (Wiley Publishing); which are incorporated herein by reference.

Fragments or analogs comprising substantially one or more regions of the N-terminal regulatory domain of ASK1 may be fused to heterologous polypeptide sequences, wherein the resultant fusion protein exhibits the functional property(ies) conferred by the ASK1 fragment. Alternatively, ASK1 polypeptides wherein one or more functional domain have been deleted will exhibit a loss of the property normally conferred by the missing fragment. Similar fragments or deletions may also be made for Raf-1.

By way of example and not limitation, an amino acid sequence conferring the property of binding to Raf-1 may be fused to β-galactosidase to produce a fusion protein that can bind an immobilized ASK1 polypeptide in a binding reaction and which can enzymatically convert a chromogenic substrate to a chromophore.

Although one class of preferred embodiments are fragments having amino- and/or carboxy-termini corresponding to amino acid positions near functional domains borders, alternative ASK1 and/or Raf-1 fragments may be prepared. The choice of the amino- and carboxy-termini of such fragments rests with the discretion of the practitioner and will be made based on experimental considerations such as ease of construction, stability to proteolysis, thermal stability, immunological reactivity, amino- or carboxyl-terminal residue modification, or other considerations.

Peptidomimetics

In addition to ASK1 polypeptides consisting only of naturally occurring amino acids, Raf-1 binding peptidomimetics that share sufficient structural homology with any one of the amino acid sequences provided in SEQ ID NOS:3, 5, 7, 8, 10, and 11 are also contemplated. For example, peptidomimetics of a polypeptide comprising amino acid residues 69-110 of SEQ ID NO:1 (set forth in SEQ ID NO:11) may be suitable as drugs for inhibition of ASK1-mediated cell death.

Peptide analogs are commonly used in the pharmaceutical industry as non-peptide drugs with properties analogous to those of the template peptide. These types of non-peptide compound are termed “peptide mimetics” or “peptidomimetics” (Fauchere (1986) Adv. Drug Res. 15:29; Veber and Freidinger (1985) TINS, p. 392; and Evans et al. (1987) J. Med. Chem 30:1229; which are incorporated herein by reference) and are usually developed with the aid of computerized molecular modeling. Peptide mimetics that are structurally similar to therapeutically useful peptides may be used to produce an equivalent therapeutic or prophylactic effect.

Generally, peptidomimetics are structurally similar to a paradigm polypeptide (i.e., a polypeptide that has a biological or pharmacological activity), such as the Raf-1 binding peptides derived from the N-terminal regulatory domain of human ASK1 that are presented herein as SEQ ID NOS:2-11, but have one or more peptide linkages optionally replaced by a linkage selected from the group consisting of: —CH₂NH—, —CH₂S—, —CH₂ —CH₂—, —CH.dbd.CH— (cis and trans), —COCH₂—, —CH(OH)CH₂—, and —CH₂SO—, by methods known in the art and further described in the following references: Spatola (1983) Chemistry and Biochemistry of Amino Acids, Peptides, and Proteins, ed. Weinstein (Marcel Dekker), New York; Spatola, (March 1983) Vega Data 1:3, “Peptide Backbone Modifications” (general review); Morley (1980) Trends Pharm. Sci. pp. 463-468 (general review); Hudson et al. (1979) Int. J. Pept. Prot. Res. 14:177-185 (—CH₂NH—, CH₂ CH₂—); Spatola et al. (1986) Life Sci. 38:1243-1249 (—CH₂ —S); Hann (1982) J. Chem. Soc. Perkin Trans. I 307-314 (—CH—CH—, cis and trans); Almquist et al. (1980) J. Med. Chem. 23:1392-1398 (—COCH.sub.2-); Jennings-White et al. (1982) Tetrahedron Lett. 23:2533 (—COCH₂—); Szelke et al. (1982) European Appln. EP 45665 CA:97:39405 (—CH(OH)CH₂—); Holladay et al. (1983) Tetrahedron Lett. 24:4401-4404 (—C(OH)CH₂—); and Hruby (1982) Life Sci. 31:189-199 (—CH₂—S—); each of which is incorporated herein by reference.

A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and the like. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch (1992) Ann. Rev. Biochem. 61:387, incorporated herein by reference).

The amino acid sequence of an ASK1 polypeptide that is capable of disrupting the protein-protein interaction between Raf-1 and ASK1 will enable those of skill in the art to produce or identify agents that are structurally similar to the disclosed ASK1 peptide sequences and variants thereof.

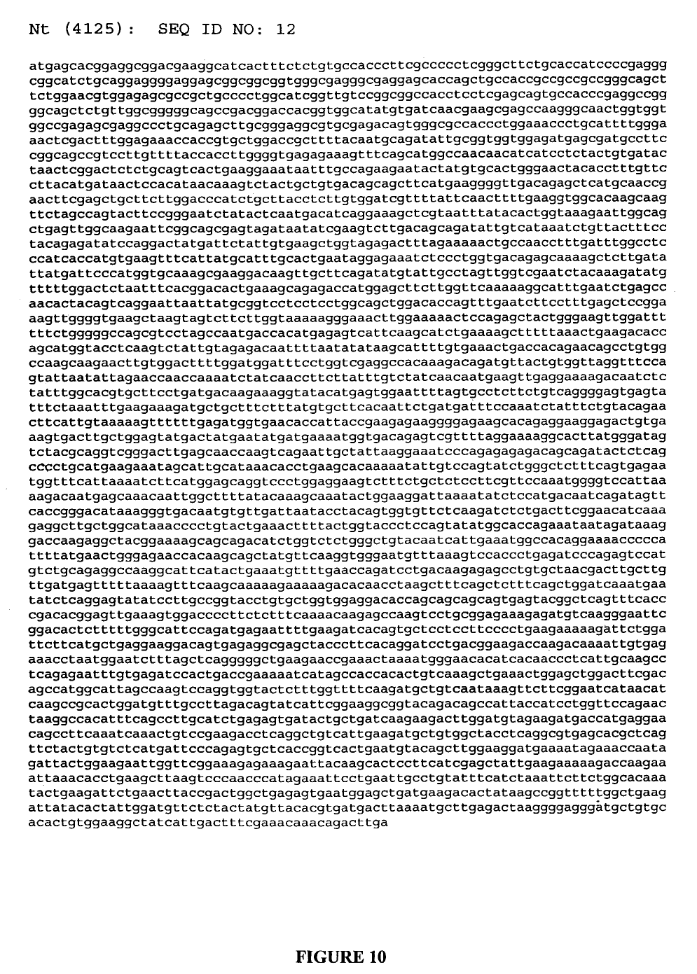

Particularly useful polypeptides include a recombinant or synthetic polypeptide comprising an amino acid sequence set forth in SEQ ID NOS:3, 5, 7, 8, 10, and 11. SEQ ID NO:3 corresponds to amino acid residues 6 to 230 of the full-length human ASK1 sequence set forth in SEQ ID NO:1. SEQ ID NO:5 corresponds to amino acid residues 6 to 430 of the full-length human ASK1 sequence set forth in SEQ ID NO:1. SEQ ID NO:7 corresponds to amino acid residues 6 to 163 of the full-length human ASK1 sequence set forth in SEQ ID NO:1. SEQ ID NO:8 corresponds to amino acid residues 69 to 230 of the full-length human ASK1 sequence set forth in SEQ ID NO:1. SEQ ID NO:10 corresponds to amino acid residues 69 to 163 of the full-length human ASK1 sequence set forth in SEQ ID NO:1, and SEQ ID NO:11 corresponds to amino acid residues 69 to 119 of the full-length human ASK1 sequence set forth in SEQ ID NO:1. Such polypeptides may be produced in prokaryotic or eukaryotic host cells by expression of polynucleotides encoding an ASK1 peptide sequence, frequently as part of a larger polypeptide (e g., a fusion peptide). Suitable polynucleotide sequences can be back translated from one of the disclosed amino acid sequences or determined from the nucleic acid sequence of ASK1 presented in FIG. 10 as SEQ ID NO:12. Alternatively, suitable peptides may be synthesized by chemical methods.

Methods for Rational Drug Design

ASK1 polypeptides, especially those portions that form direct contacts in the ASK1/Raf-1 interaction disclosed herein, can be used for rational drug design of candidate ASK1-modulating agents (e.g., apoptosis modulators and immunomodulators). The substantially purified ASK1/Raf-1 complexes and the identification of Raf-1 as a binding partner for ASK1 provided herein permits production of substantially pure polypeptide complexes and computational models which can be used for protein X-ray crystallography or other structure analysis methods, such as the DOCK program (Kuntz et al. (1982) J. Mol. Biol. 161:269; Kuntz (1992) Science 257:1078) and variants thereof. Potential therapeutic drugs may be designed rationally on the basis of structural information thus provided. In one embodiment, such drugs are designed to prevent formation of an ASK1/Raf-1 complex. Thus, the present invention may be used to design drugs, including drugs with a capacity to modulate ASK1-mediated cell signaling and apoptosis.

The design of compounds that interact preferentially with a Raf-1 polypeptide or an ASK1/Raf-1 complex can be developed using computer analysis of three-dimensional structures. A set of molecular coordinates can be determined using: (1) crystallographic data; (2) data obtained by other physical methods; (3) data generated by computerized structure prediction programs operating on the deduced amino acid sequence data; or, preferably, a combination of these data. Examples of physical methods that may be used to define structure are known in the art.

It is not intended that the present invention be limited by the particular method used to obtain structural information. Furthermore, it is not intended that the present invention be limited to a search for any one type of drug; one or more of the molecules may be naturally occurring, produced by recombinant methods or may be synthetic, or may be a chemically modified form of a naturally occurring molecule.

In some embodiments, it is desirable to compare the structure of ASK1 or Raf-1 polypeptides(s) to the structure(s) of other proteins. This will aid in the identification of and selection of drugs that either selectively interact with Raf-1 or ASK1, or have a broad-spectrum effect on more than one species of related polypeptide (e.g., other Raf-1 related proteins). Production and Applications of Anti-ASK1 Antibodies

Native ASK1 peptides, or fragments or analogs thereof, may be used to immunize an animal for the production of specific antibodies. These antibodies may comprise a polyclonal antiserum or may comprise a monoclonal antibody produced by hybridoma cells. For general methods to prepare antibodies see Harlow and Lane (1988) Antibodies: A Laboratory Manual (Cold Spring Harbor Laboratory, Cold Spring Harbor, N.Y.), which is incorporated herein by reference.

For example, but not for limitation, a recombinantly produced ASK1 peptide such as a peptide comprising the amino acid sequence set forth in SEQ ID NO:11 can be injected into a mouse along with an adjuvant following immunization protocols known to those of skill in the art so as to generate an immune response. Animals other than mice and rats may be used to raise antibodies; for example, goats, rabbits, sheep, and chickens may also be employed to raise antibodies reactive with an ASK1 or Raf-1 protein.

Transgenic mice having the capacity to produce substantially human antibodies also may be immunized and used for a source of anti-ASK1 antiserum and/or for making monoclonal-secreting hybridomas.

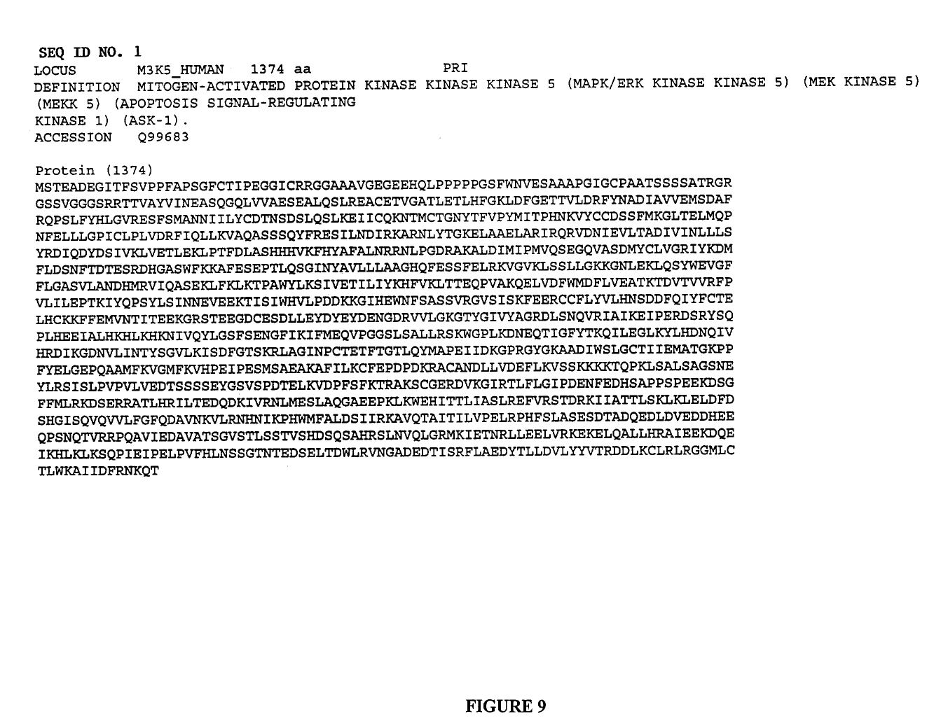

Typically, approximately at least 1-50 μg of a peptide or analog is used for the initial immunization, depending upon the length of the polypeptide. Alternatively or in combination with a recombinantly produced ASK1 polypeptide, a chemically synthesized peptide having an ASK1 sequence may be used as an immunogen to raise antibodies which bind human ASK1, such as the native human polypeptide having the sequence shown essentially in FIG. 9 (SEQ ID NO:1).

Immunoglobulins that bind the recombinant fragment may be harvested from the immunized animal as an antiserum, and may be further purified by immunoaffinity chromatography or other means. Additionally, spleen cells may be harvested from the immunized animal (typically rat or mouse) and fused to myeloma cells to produce a bank of antibody-secreting hybridoma cells. This bank of hybridomas may be screened for clones that secrete immunoglobulins that bind the recombinantly produced ASK1 polypeptide (or chemically synthesized ASK1 polypeptide).

Bacteriophage antibody display libraries may also be screened for binding to a ASK1 polypeptide, such as the N-terminal regulatory region of human ASK1 protein, or a fusion protein comprising one of the amino acid sequences set forth in SEQ ID NOS:3, 5, 7, 8, 10 and 11. Generally speaking, an ASK1 polypeptide sequence sufficient to comprise an ASK1 epitope will comprise at least 3-5 contiguous amino acids. Generally such peptides and the fusion protein portions consisting of ASK1 sequences for screening antibody libraries comprise about at least 3 to 5 contiguous amino acids of ASK1, frequently at least 7 contiguous amino acids of ASK1, usually comprise at least 10 contiguous amino acids of ASK1, and most usually comprise a sequence of at least 14 contiguous amino acids.

Combinatorial libraries of antibodies have been generated in bacteriophage lambda expression systems that may be screened as bacteriophage plaques or as colonies of lysogens (Huse et al. (1989) Science 246:1275; Caton and Koprowski (1990) Proc. Natl. Acad. Sci. (USA) 87:6450; Mullinax et al. (1990) Proc. Natl. Acad. Sci. U.S.A. 87:8095; Persson et al. (1991) Proc. Natl. Acad. Sci. USA 88:2432). Various embodiments of bacteriophage antibody display libraries and lambda phage expression libraries have been described (Kang et al. (1991) Proc. Natl. Acad. Sci. USA 88:4363; Clackson et al. (1991) Nature 352:624; McCafferty et al. (1990) Nature 348:552; Burton et al. (1991) Proc. Natl. Acad. Sci. USA 88:10134; Hoogenboom et al. (1991) Nucleic Acids Res. 19:4133; Chang et al. (1991) J. Immunol. 147:3610; Breitling et al. (1991) Gene 104:147; Marks et al. (1991) J. Mol. Biol. 222:581; Barbas et al. (1992) Proc. Natl. Acad. Sci. USA 89:4457; Hawkins and Winter (1992) J. Immunol. 22:867; Marks et al. (1992) Biotechnology 10:779; Marks et al. (1992) J. Biol. Chem. 267:16007; Lowman et al. (1991) Biochemistry 30:10832; Lerner et al. (1992) Science 258:1313, incorporated herein by reference). Typically, a bacteriophage antibody display library is screened with an ASK1 Raf-1 binding polypeptide that is immobilized (e.g., by covalent linkage to a chromatography resin to enrich for reactive phage by affinity chromatography) and/or labeled (e.g., to screen plaque or colony lifts).

Alternatively, a monoclonal antibody specific for an epitope present in one of the Raf-1 binding N-terminal ASK1 peptides disclosed herein that neutralizes the prosurvival effects of Raf-1 can be used to identify immunologically cross-reactive agents that represent candidate agents for modulating ASK1-dependent cellular function.

ASK1 peptides that are useful as immunogens, or for screening a bacteriophage antibody display library, are suitably obtained in substantially pure form, that is, typically about 50 percent (w/w) or more purity, substantially free of interfering proteins and contaminants. Preferably, these polypeptides are isolated or synthesized in a purity of at least 80 percent (w/w) and, more preferably, in at least about 95 percent (w/w) purity, being substantially free of other proteins of humans, mice, or other contaminants.

For some applications of these antibodies, such as identifying immunocrossreactive proteins, the desired antiserum or monoclonal antibody(ies) is/are not monospecific. In these instances, it may be preferable to use a synthetic or recombinant fragment of one of the ASK1 N-terminal truncation peptides demonstrated herein to bind to Raf-1 as an antigen rather than using the entire native protein. More specifically, where the object is to identify immunocrossreactive polypeptides that comprise a particular structural moiety, such as a Raf-1 binding domain, it is preferable to use as an antigen a fragment corresponding to part or all of a commensurate structural domain in the ASK1 protein.

If antiserum is raised to an ASK1 fusion polypeptide, such as a fusion protein comprising a ASK1 immunogenic epitope fused to 13-galactosidase or glutathione S-transferase, the antiserum is preferably preadsorbed with the non-ASK1 fusion partner (e.g, β-galactosidase or glutathione S-transferase) to deplete the antiserum of antibodies that react (i.e., specifically bind to) the non-ASK1 portion of the fusion protein that serves as the immunogen.

Methods of Identifying Protein Interaction Inhibitors and Apoptosis-Modulating Agents

The invention provides efficient methods of identifying agents, compounds, or lead compounds for agents active at the level of modulating ASK1-dependent cellular functions such as apoptosis. Generally, these screening methods involve assaying for compounds that either inhibit or modulate a binding interaction between an N-terminal region of ASK1 and a Raf-1 binding target. A wide variety of assays for modulatory/binding agents are provided including labeled in vitro protein-protein binding assays, cell based assays, immunoassays, apoptosis assays (e.g. Kreider et al. (1992) Science 255:1700-1702), etc. The methods are amenable to automated, cost-effective, high throughput screening of chemical libraries for lead compounds.

In vitro binding assays employ a mixture of components including an ASK1 polypeptide, or an N-terminal fragment thereof, which may be part of a fusion product with another peptide or polypeptide, e.g. a tag for detection or anchoring, etc. The assay mixtures further comprise a Raf-1 binding target protein. While native binding targets may be used, it is frequently preferred to use portions thereof as long as the portion provides binding affinity and avidity to the subject ASK1 polypeptide conveniently measurable in the assay. In the context of screening, an in vitro binding assay mixture may also comprise a candidate modulatory (e.g., antagonist or agonist) agent. Candidate agents encompass numerous chemical classes, though typically they are peptides, peptidomimetics, organic compounds, or, preferably, small organic compounds; these compounds may be obtained from a wide variety of sources, including libraries of synthetic or natural compounds. A variety of other reagents such as salts, buffers, neutral proteins (e.g. albumin, detergents, protease inhibitors, nuclease inhibitors, antimicrobial agents), etc. may also be included. The mixture components can be added in any order that provides for the requisite bindings, and incubations may be performed at any temperature that facilitates optimal binding. The mixture is incubated under conditions whereby, but for the presence of the candidate pharmacological agent, the ASK1 polypeptide specifically binds the Raf-1 binding target. Incubation periods are generally chosen for optimal binding, but may also be minimized to facilitate rapid, high-throughput screening.

After incubation, the agent-biased binding between the ASK1 polypeptide and the Raf-1 binding target is detected by any convenient way. For cell-free binding assays, a separation step is often used to separate bound from unbound components. Separation may be effected by precipitation, immobilization, etc., followed by washing by, for example, membrane filtration, gel chromatography. For cell-free binding assays, one of the components usually comprises or is coupled to a label. The label may provide for direct detection via radioactivity, fluorescence, luminescence, optical or electron density, etc., or indirect detection via an epitope tag, an enzyme, etc. A variety of methods may be used to detect the label, depending on the nature of the label and other assay components, e.g., through optical or electron density, radiative emissions, nonradiative energy transfers, etc., or indirectly detected with antibody conjugates, etc. A difference in the binding activity of the ASK1 polypeptide to the Raf-1 target in the absence of the agent as compared with the binding activity in the presence of the agent indicates that the agent modulates the ASK1/Raf-1 binding interaction. A difference, as used herein, is statistically significant and preferably represents at least a 50%, more preferably at least a 90% difference.

Fluorescent Polarization Assays

Fluorescent polarization (FP) assays can be used to detect binding interactions and to identify agents that modulate (e.g., inhibit or promote) a particular binding interaction. Generally speaking, an FP assay is capable of detecting a reaction product in the presence of a reaction substrate, despite the fact that the product is detected by virtue of a fluorescent label that it is also present on the reaction substrate. In performing a typical fluorescent binding assay, a typically small fluorescently labeled molecule, for example an ASK1 peptide, is used to bind to a much larger molecule, for example a Raf-1 polypeptide.

Generally speaking the small fluorescently labeled molecule has a relatively fast rotational correlation time relative to the much slower rotational correlation time that characterizes the larger molecule. The binding of the fluorescently labeled small molecule to the larger molecule significantly increases the rotational correlation time (i.e., decreases the amount of rotation) of the resulting labeled complex over that of the free unbound labeled reaction substrate. This has a corresponding effect on the level of polarization that is detectable. More specifically, the labeled complex presents much higher fluorescent polarization than the unbound small molecule.

In the context of a FP binding assay suitable for detecting a protein-protein binding interaction, the assay is usually configured such that the first reagent (which typically bears a fluorescent label) is contacted with a second reagent, which binds to the first reagent to yield a fluorescently labeled product. Typically, the second reagent is characterized by a level of charge such that the product resulting from the binding interaction has a charge that is substantially different from that of the first reagent alone.

FP assays provide a flexible screening assay for the identification of potential modulators, inhibitors, enhancers, agonists, or antagonists of the binding interaction in question. In the context of a screening assay which comprises a mixture comprising a small fluorescently labeled first molecule, for example a labeled ASK1 peptide, a second full-length Raf-1 polypeptide, and a candidate agent (e.g., potential modulator, inhibitor, enhancer, agonsist or antagonist), the fluorescent polarization of the of the reaction mixture is compared in the presence and absence of candidate agents to determine whether the agents have any effect on the binding interaction of interest. In particular, in the presence of inhibitors of the binding interaction, the fluorescent polarization will decrease, as more free-labeled ligand is present in the assay sample. Conversely, in the presence of an enhancer of the binding interaction an increase in fluorescent polarization will result, as more complexed (e.g., bound) and less free-labeled ligand is present in the assay sample.

The fluorescent label on the first molecule can be selected from any of a variety of different flurochromes. Typically, fluorescein or rhodamine derivatives are well suited for use in a FP binding assay. A variety of detection schemes that can be employed to detect the rate of rotation of a molecule, such as nuclear magnetic resonance spectroscopy, electron spin resonance spectroscopy, and triplet state absorbance anisotropy.

A fluorescent polarization assay using a fluorescence labeled ASK1 peptide and unlabeled Raf-1 polypeptide can be used to screen for agents (e.g., small molecules, peptides, peptidomimetics) that interfere with the fluorescent signal that results from the binding interaction between peptides comprising the minimal binding sites necessary to mediate the interaction. Suitable ASK1 peptides for use in a FP binding assay capable of identifying agents that modulate ASK1-mediated apoptosis include a peptide comprising the Raf-1 binding site present in one of the amino acid sequences set forth in SEQ ID NOS:3, 5, 7, 8, 10 and 11. Alternatively, a smaller peptide comprising a fragment of one of the above-identified peptides that defines the minimal Raf-1 binding sequence can be used to establish a suitable FP binding assay. It is well within the skill of a practitioner to utilize the information provided herein in combination with the teachings set forth in U.S. Pat. No: 6,287,774, WO 95/15981, or WO 99/64840, the teachings of which are incorporated herein by reference, to design a suitable FP screening assay.

Yeast Two-Hybrid Screening Assays

Transcriptional activators are proteins that positively regulate the expression of specific genes. They can be functionally dissected into two structural domains: one region that binds to specific DNA sequences and thereby confers specificity, and another region termed the activation domain that binds to protein components of the basal gene expression machinery (Ma and Ptashne (1988) Cell 55:443). These two domains must be physically connected in order to function as a transcriptional activator. Two-hybrid systems exploit this finding by joining an isolated DNA binding domain to one protein (protein X), while joining the isolated activation domain to another protein (protein Y). When X and Y interact to a significant extent, the DNA binding and activation domains will now be connected, and the transcriptional activator function reconstituted (Fields and Song (1989) Nature 340:245).

In a two-hybrid system, the yeast host strain is engineered so that the reconstituted transcriptional activator drives the expression of a specific reporter gene such as HIS3 or lacZ, which provides the read-out for the protein-protein interaction. One advantage of two-hybrid systems for monitoring protein-protein interactions is their sensitivity in detection of physically weak, but physiologically important, protein-protein interactions. As such, these systems offer a significant advantage over other methods for detecting protein-protein interactions (e.g., ELISA assays).

One such two-hybrid system used to identify polypeptide sequences which bind to a predetermined polypeptide sequence involves a system where the predetermined polypeptide sequence is present in a fusion protein (Chien et al. (1991) Proc. Natl. Acad. Sci. USA 88:9578). This approach identifies protein-protein interactions in vivo through reconstitution of a yeast Gal4 transcriptional activator protein (Fields and Song (1989) Nature 340:245). Typically, the method is based on the properties of the yeast Gal4 protein, which consists of separable domains responsible for DNA-binding and transcriptional activation. Polynucleotides encoding two hybrid proteins are constructed and introduced into a yeast host cell, where one polynucleotide consists of the yeast Gal4 DNA-binding domain fused to a polypeptide sequence of a known protein (e.g., minimal Raf-1 binding domain of ASK1) and the other consists of the Gal4 activation domain fused to a polypeptide sequence of a second protein (e.g., Raf-1 binding target).

Intermolecular binding between the two fusion proteins reconstitutes the Gal4 DNA-binding domain with the Gal4 activation domain, which leads to the transcriptional activation of a reporter gene (e.g., lacZ, HIS3) which is operably linked to a Gal4 binding site. Typically, the two-hybrid method is used to identify novel polypeptide sequences which interact with a known protein (Silver and Hunt (1993) Mol. Biol. Rep. 17:155; Durfee et al. (1993) Genes Devel. 7; 555; Yang et al. (1992) Science 257:680; Luban et al. (1993) Cell 73:1067; Hardy et al. (1992) Genes Devel. 6; 801; Bartel et al. (1993) Biotechniques 14:920; and Vojtek et al. (1993) Cell 74:205).

Variations of the two-hybrid method have been used to identify mutations of a known protein that affect its binding to a second known protein (Li and Fields (1993) FASEB J. 7:957; Lalo et al. (1993) Proc. Natl. Acad. Sci. USA 90:5524; Jackson et al. (1993) Mol. Cell. Biol. 13; 2899; and Madura et al. (1993) J. Biol. Chem. 268:12046). Therefore, the assay provides an alternative to the coprecipitation-based immunoassays described herein. This variation of the yeast two-hybrid assay system provides a convenient assay for determining the effect of either randomly or selectively introduced point mutations in the Raf-1 binding fragment of the N-terminal regulatory domain of ASK1 (e.g., amino acids 6-230 or SEQ ID NO:1), and allows for the identification of mutations that disrupt or promote the binding interaction. Identification of such residues may facilitate definition of the minimal ASK1 binding site necessary to mediate Raf-1 binding. Alternatively, the two-hybrid method also provides a method for performing an alanine scan to identify the minimal binding site. The results of mutagenesis studies can complement the results obtained using the conventional deletion analysis performed herein.

Each of these two-hybrid methods rely upon a positive association between two Gal4 fusion proteins, thereby reconstituting a functional Gal4 transcriptional activator, which then induces transcription of a reporter gene operably linked to a Gal4 binding site. Transcription of the reporter gene produces a positive readout, typically manifested as an enzyme activity (e.g., β-galactosidase). A positive readout condition is generally identified as one or more of the following detectable conditions: (1) an increased transcription rate of a predetermined reporter gene; (2) an increased concentration or abundance of a polypeptide product encoded by a predetermined reporter gene, typically such as an enzyme which can be readily assayed in vivo; and/or (3) a selectable or otherwise identifiable phenotypic change in an organism (e.g., yeast) harboring the reverse two-hybrid system. Generally, a selectable or otherwise identifiable phenotypic change that characterizes a positive readout condition confers upon the organism either: a selective growth advantage on a defined medium, a mating phenotype, a characteristic morphology or developmental stage, drug resistance, or a detectable enzymatic activity (e.g., β-galactosidase, luciferase, alkaline phosphatase, and the like).

The invention also provides host organisms (typically a unicellular organism) which harbor an ASK1-related protein two-hybrid system, typically in the form of polynucleotides encoding a first hybrid protein, a second hybrid protein, and a reporter gene, wherein said polynucleotide(s) are either stably replicated or introduced for transient expression. In one embodiment, the host organism is a yeast cell (e.g., Saccharomyces cervisiae) in which the reporter gene transcriptional regulatory sequence comprises a Gal4-responsive promoter.

“Reverse” two-hybrid systems allow a practitioner to select for mutations, agents or competitive inhibitors that disrupt two-hybrid interactions. One such system employs the gene URA3 as a reporter, and is based on the fact that, because expression of URA3 is toxic to cells grown on 5-fluoroorotic acid, a two-hybrid interaction will result in cell death. Therefore, dissociation or inhibition of the interaction will lead to a loss of URA3 expression, thereby allowing cell growth (Drees (1999) Current Opinion in Chem. Bio.3:64-70; Vidal (1996) Proc. Natl. Acad. Sci. USA 93:10315-10326). Huang and Schreiber, the teachings of which are incorporated herein by reference, have described a miniaturized high-throughput screening method based on a reverse two-hybrid scheme to create a system that is capable of identifying small inhibitors of protein-protein interactions in nanodroplets (Huang and Schreiber Proc Nat. Acad. Sci. USA, 94:13396-13401).

Apoptosis Assays

Numerous apoptosis assays can be used to identify agents that modulate human apoptosis signal-regulating kinase 1 (ASK1)-mediated cell death. Numerous techniques capable of defining the functional endpoint of apoptosis can be found in the literature, or in, for example, Darzynkiewicz et al. (1997) Cytometry 27:1-20; Ormerod (1998) Leukemia 12:1013-1025; Bedner et al. (1999) 35:181-195; Sgonc and Gruber (1998) J. Exp. Gerontol. 33:525-533. Apoptosis methods include morphological examination for characteristic cellular changes including nuclear fragmentation and formation of apoptotic bodies, the detection of apoptosis-related DNA degradation by measuring DNA laddering, determining the percentage of sub-G1 cells after staining with propidium iodide, and DNA break formation by nick end labeling by TUNEL.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

The following examples are presented by way of illustration, not by way of limitation.

Plasmids

Expression vectors for ASK1 and its mutants have been described (Ichijo et al. (1997) Science 275:90-94; Zhang et al. (1999) Proc. Natl. Acad. Sci. USA 96:8511-8515). Wild-type (WT) MEK1^{WT}, constitutively active mutant MEK1^{C}, and dominant negative mutant MEK1^{dn} were gifts from K. Guan, Univ. of Michigan (Sugimoto et al. (1998) EMBO J. 17:1717-1727). pcDNA3-FLAG-Raf-1, Raf-N, and Raf-C have been described (Zhang et al. (1997) J. Biol. Chem. 272:13717-13724). Mutations Raf^{K375W} and Raf^{S259A/S621A} were generated by using the QuikChange site-directed mutagenesis kit (Stratagene) with pcDNA3-FLAG-Raf-1 as a template. Hemagglutinin (HA)-ASK1 NT (6-678) and ΔC(6-936) were generated by PCRs and subcloned into pcDNA3. HA-ASK1 ΔN (678-1375) was constructed in the Gateway cloning expression vector pDEST26 (Invitrogen). pcDNA3-HA-ASK1 ΔK (1-819/1057-1375) was generated by digestion of pcDNA3-HA-ASK1 with BamHI and BglII and religation. HA-ASK1-CT (1071-1375) was constructed by EcoRI digestion and religation of pcDNA3-HA-ASK1.

Cell Culture and DNA Transfection

HeLa, COS7, and L929 cells were cultured in DMEM (Mediatech, Washington, D.C.) with 10% FBS (Atlanta Biologicals, Norcross, Ga.). Transfection was performed with FuGENE 6 (Roche Molecular Biochemicals) according to the manufacturer's instructions.

Apoptosis Assays

For the nuclear morphology assays (Zhang et al. (1999) Proc. Natl. Acad. Sci. USA 96:8511-8515), 2×10⁵ HeLa cells were cultured in 35-mm dishes containing glass coverslips. Cells were cotransfected with pTJM9 (0.4 μg) encoding enhanced green fluorescent protein (eGFP) and test plasmids (1.6 μg total) or supplemented with pcDNA3. Eighteen hours after transfection, the medium was changed to serum-free DMEM. Twenty-four hours later, cells on the glass coverslips were washed, fixed (0.5% glutaraldehyde/2% formaldehyde in PBS), stained with 4′,6-diamidino-2-phenylindole (DAPI) in Vectashield mounting medium (Vector Laboratories), and visualized by using a fluorescence microscope as described (Zhang et al. (1999) Proc. Natl. Acad. Sci. USA, 96:8511-8515). Transfected cells with fragmented nuclei were scored for apoptosis in a blind fashion. Cells remaining in the dishes were lysed and immunoblotted with various antibodies by using the ECL system (Amersham Pharmacia). For the β-galactosidase (β-gal)-based cell morphology assay, 2×10⁵ HeLa cells were cultured in 35-mm plates and cotransfected with a lacZ expression vector (0.4 μg) and test plasmids (1.6 μg total). Twenty-four hours after transfection, cells were fixed, stained with 5-bromo-4-chloro-3-indolyl-β-D-galactopyranoside, and scored for apoptosis as described (Chang et al. (1998) Science 281:1860-1863). Parallel samples were collected for Western blotting.

The DNA content-based apoptosis assay was performed with a fluorescence-activated cell sorter as described (Spector et al. (1997) Cells: A Laboratory Manual (Cold Spring Harbor Lab. Press, Plainview, N.Y.). Briefly, COS7 cells (2×10⁵) were cotransfected with pEGFP-F (0.4 μg; CLONTECH) encoding a farnesylated eGFP and test plasmids (1.6 μg total). Twenty-four hours after transfection, total cells were trypsinized, resuspended in PBS, fixed in ice-cold ethanol followed by overnight incubation, treated with RNase A (Sigma-Aldrich), and stained with propidium iodide (Sigma-Aldrich). Samples were analyzed with a FACSort flow cytometer (Becton Dickinson). The DNA content of transfected cells was determined by using WINMDI 2.8 software (Trotter, Scripps Research Institute, La Jolla, Calif.).

Immunoprecipitation and Western Blotting

Forty-eight hours after transfection, 4×10⁵ COS7 cells were lysed in 300 μl of lysis buffer (0.2% Nonidet P-40/10 mM Hepes, pH 7.4/150 mM NaCl/5 mM NaF/2 mM Na₃VO_{4/5} mM Na₄P₂O₇/10 μg/ml aprotonin/10 μg/ml leupeptin/1 mM phenylmethylsulfonyl fluoride). Cell extracts were clarified by centrifugation and used for immunoprecipitation with various antibodies and protein G-Sepharose (Amersham Pharmacia). Immunocomplexes were washed four times with lysis buffer containing 1% Nonidet P-40 or RIPA buffer (1% Nonidet P-40/0.5% sodium deoxycholate/0.1% SDS/137 mM NaCl/20 mM Tris.HCl, pH 7.5) and resolved on SDS/PAGE (12.5%) for Western blotting. The enzyme-linked immunoblotting procedures were performed essentially as described (Zhang et al. (1999) Proc. Natl. Acad. Sci. USA 96:8511-8515). Corresponding secondary antibodies were used against each primary antibody: horseradish peroxidase-conjugated goat anti-mouse IgG for monoclonal antibodies and horseradish peroxidase-conjugated goat anti-rabbit IgG for polyclonal antibodies (Santa Cruz Biotechnology). Cross-reacting materials were visualized by using the ECL detection reagents (Amersham Pharmacia).

### Example 1

Overexpression of Raf-1 Inhibits ASK1-Induced Apoptosis

To investigate the role of the Raf-1 pathway in suppressing apoptotic signaling, we tested the effect of Raf-1 on ASK1-induced apoptosis. HeLa cells were transiently transfected with HA-tagged human ASK1 alone or together with FLAG-tagged Raf-1.

HeLa cells were transfected with the plasmids pcDNA3-HA-ASK1 (1.2 μg) or pcDNA3-FLAG-Raf-1 (0.4 μg) along with an eGFP expression vector (0.4 μg) as indicated. Eighteen hours posttransfection, cells were placed in serum-free medium for an additional 24 h before staining with DAPI. Nuclear morphology of transfected cells was examined by fluorescence microscopy as described (Zhang et al. (1999) Proc. Natl. Acad. Sci. USA 96:8511-8515).

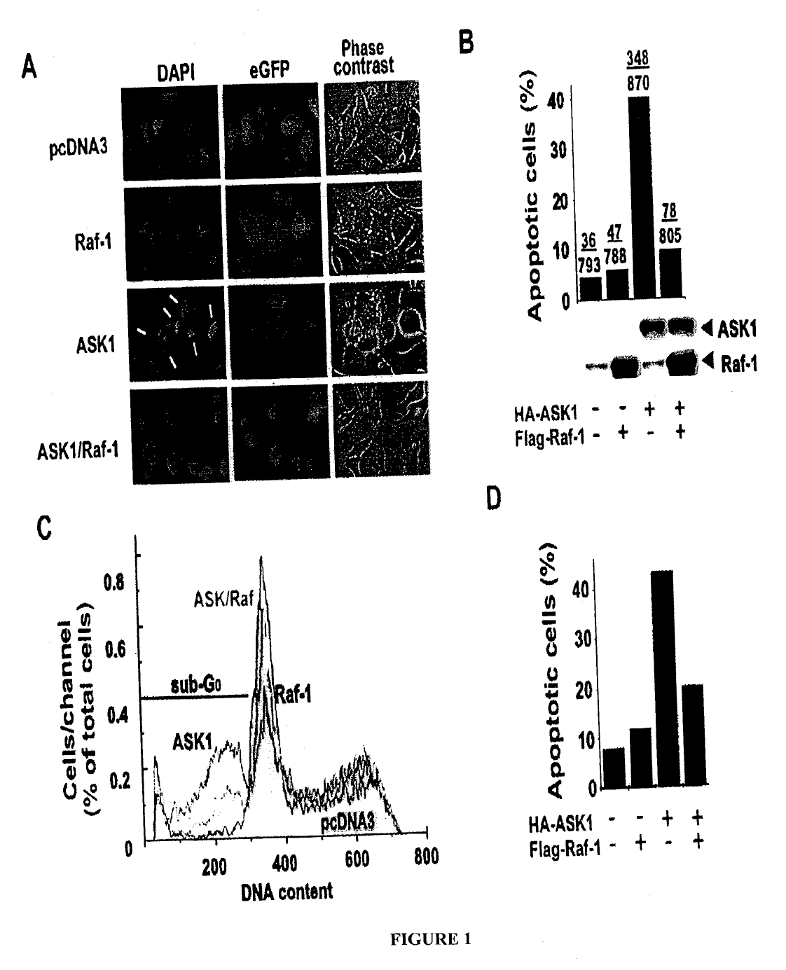

Apoptotic cell death was monitored by nuclear morphology. Cells transfected with the control vector or a vector encoding Raf-1 showed homogenous DAPI staining and normal nuclear morphology (FIG. 1A). In contrast, expression of ASK1 induced the appearance of condensed chromatin and fragmented nuclei characteristic of apoptosis consistent with previously published data (Zhang et al. (1999) Proc. Natl. Acad. Sci. USA 96:8511-8515). FIG. 1A provides a set of photomicrographs illustrating the results of a transient transfection experiment that was performed to evaluate the effects of Raf-1 on ASK1-mediated apoptosis. Apoptotic cell death was monitored by nuclear morphology. The data indicate that coexpression of Raf-1 and ASK1 inhibits ASK-mediated apoptosis. The fraction of transfected cells with fragmented nuclei was quantified in a blind manner.

When cells were transfected with Raf-1 together with ASK1, however, the fraction of cells with apoptotic nuclear morphology was decreased, which was quantified as shown in FIG. 1B. Briefly, the samples were subjected to SDS/PAGE and Western blotting with anti-Raf-1 (SC133; Santa Cruz Biotechnology) or anti-HA (12CA5) antibodies.

These results suggest that Raf-1 can inhibit the apoptotic activity of ASK1. This decrease did not appear to result from decreased levels of ASK1 protein (FIG. 1B Lower). Similar results were obtained with COS7 cells by using a DNA content-based flow cytometry assay. FIG. 1C provides a histogram summarizing the results of a DNA content-based flow cytometry assay to analyze COS7 cells that were transfected with the same plasmids described above along with an eGFP-F expression vector. Twenty-four hours after transfection, total cells were harvested, their DNA was stained with propidium iodide, and eGFP and propidium iodide signals were measured on a FACSort flow cytometer. Transfected cells (eGFP-positive) were placed in various phases of the cell cycle based on their DNA content. Apoptotic cells with fragmented DNA (subG₀) are indicated. FIG. 1D provides a graphic summary of the data obtained from the flow cytometric analysis. The data presented in FIG. 1D were compiled to show the amount of apoptosis caused by expression of transfected plasmids.

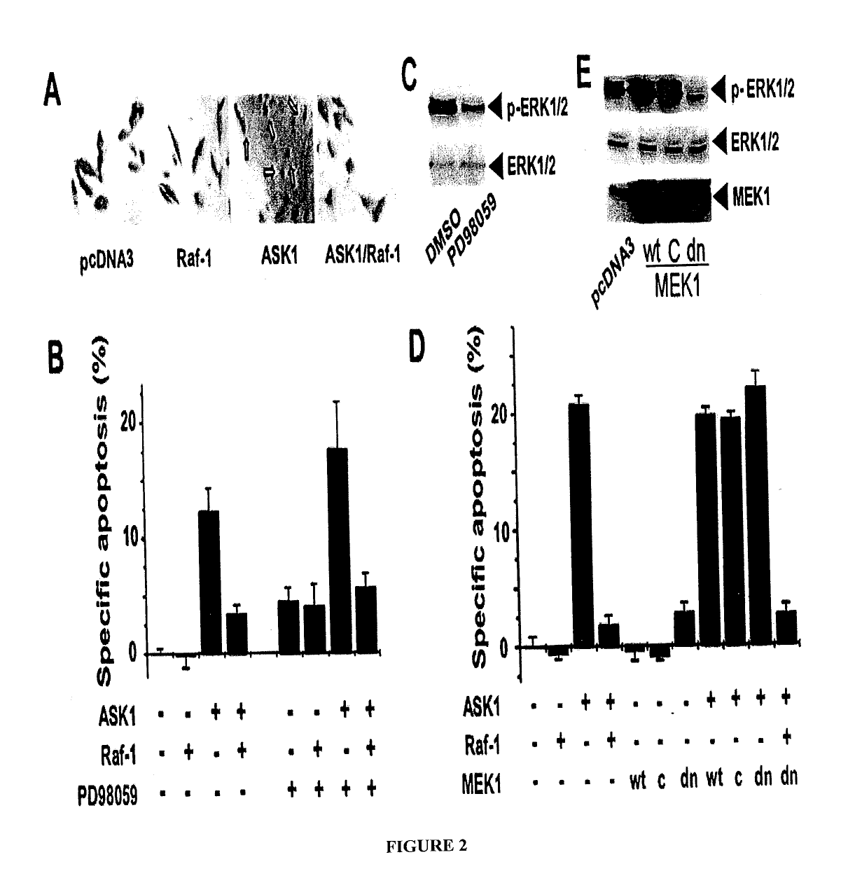

When adherent cells undergo apoptosis, they often exhibit cell shrinkage and rounded-up morphology. These features form the basis of an alternative cell morphology-based death assay (Chang et al. (1998) Science 281:1860-1863), which uses β-gal as a marker (FIG. 2A). The photomicrographs provided in FIG. 1A illustrate the morphology of HeLa cells transfected with pcDNA3, Raf-1, ASK1 and ASK1/Raf-1. All of the transfected cell types were cotransfected with a β-gal expression vector and test plasmids as indicated. Twenty-four hours after transfection, cells were fixed and stained with 5-bromo-4-chloro-3-indolyl-D-galactopyranoside. Consistent with nuclear morphology and DNA content assays, coexpression of Raf-1 diminished ASK1-induced cell death (FIG. 2B). These data suggest that a Raf-1-mediated signaling pathway may play a negative role in controlling ASK1-dependent apoptosis.

### Example 2

The MEK-ERK Pathway Is Not Required for Raf-1 to Block ASK1 Function

Raf-1-dependent activation of the MEK-ERK pathway has been shown to promote cell survival by targeting various death pathways (Le Gall et al. (2000) Mol. Biol. Cell 11:1103-1112; Bonni et al. (1999) Science 28:1358-1362; Scheid et al. (1999) J. Biol. Chem. 274:31108-31113; Shimamura et al. (2000) Curr. Biol. 10:127-135; Kurada and White (1998) Cell 95:319-329 and Bergmann et al. (1998) Cell 95:331-341). To test the hypothesis that Raf-1 regulates ASK1-induced apoptosis through the MEK-ERK pathway, we used two widely used MEK antagonists, PD98059 and U0126 (Dudley et al. (1995) Proc. Natl. Acad. Sci. USA 92:7686-7689; Favata et al. (1998) J. Biol. Chem. 273:18623-18632). Surprisingly, treatment of cells with PD98059 (60 μM) did not decrease the ability of Raf-1 to inhibit ASK1-induced cell death, although this agent diminished the activation of ERK1/2 (FIG. 2B and C). Similar results were obtained with U0126 (25 μM; data not shown). To confirm the observations with PD98059 and U0126, we used a dominant negative mutant of MEK1, MEK1_{dn}, to interfere with MEK signaling (Sugimoto et al. (1998) EMBO J. 17:1717-1727).

Expression of MEK1_{dn} decreased the basal activation level of ERK1/2 but showed no effect on the inhibition of ASK1-induced apoptosis by Raf-1 (FIG. 2D and E). However, the above MEK inhibitors are unable to completely block MEK-ERK signaling, and the remaining activity may be sufficient to transmit the Raf-1 survival signal. To test this possibility, we examined whether activation of the MEK-ERK pathway by overexpression of MEK1, an immediate effector of Raf-1, would be sufficient to mimic the Raf-1 effect and inhibit ASK1. As shown in FIG. 2E, expression of MEK1^{WT} or the constitutively active mutant MEK1C activated ERK1/2. However, neither of these MEK1 constructs was capable of attenuating the proapoptotic activity of ASK1 (FIG. 2D).

Thus, MEK1 cannot substitute for Raf-1 to inhibit ASK1 function. Because neither inhibition nor activation of MEK impacted the proapoptotic activity of ASK1, Raf-1 likely antagonizes ASK1 through a mechanism independent of the MEK-ERK pathway.

### Example 3

Raf-1 Interacts with ASK1 in Cells

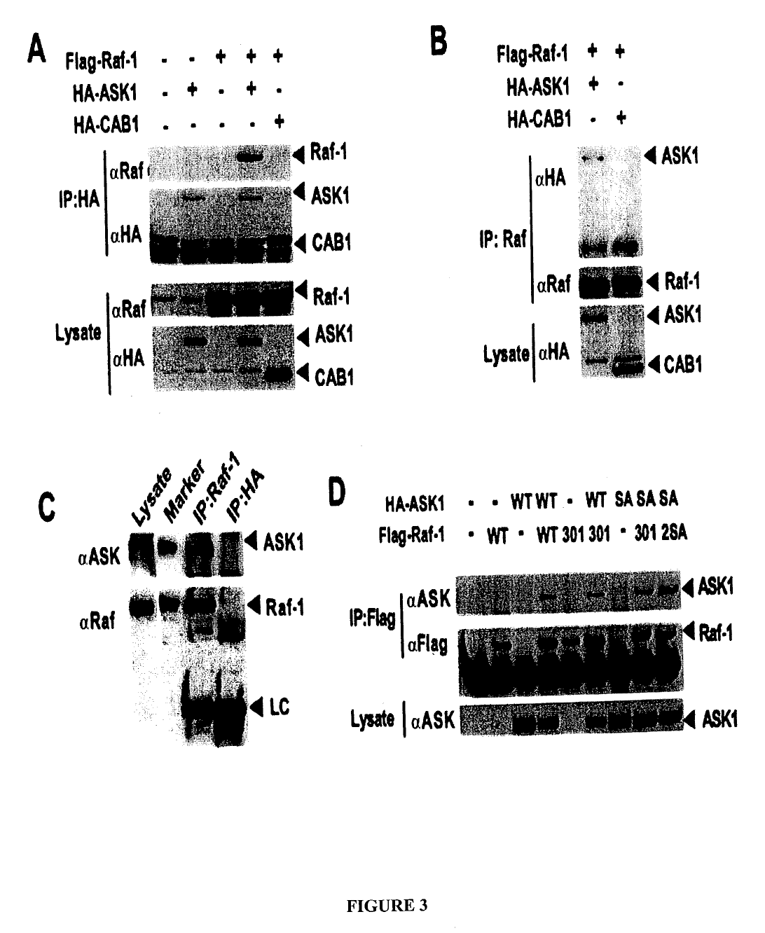

One possible mechanism for Raf-1 inhibition of ASK1 apoptotic activity is through direct interaction between the two proteins. To test this hypothesis, we performed coimmunoprecipitation experiments in COS7 cells (Zhang et al. (1999) Proc. Natl. Acad. Sci. USA, 96:8511-8515). FLAG-Raf-1 was transiently expressed with either HA-ASK1 or the negative control HA-CAB1. Immunoprecipitates were washed extensively with Nonidet P-40 (1%) lysis buffer before Western blotting with antibodies to Raf-1 and HA (Upper). HA immunocomplexes were isolated and examined. Raf-1 was found in the HA-ASK1 immunocomplex but was absent from the HA-CAB1 complex, suggesting that Raf-1 may specifically interact with ASK1 (FIG. 3A). To confirm the Raf-1/ASK1 association, we carried out reciprocal experiments by immunoprecipitating Raf-1 from COS7 cell lysates (FIG. 3B). HA-ASK1, but not HA-CAB 1, was detected in the Raf-1 immunocomplex. These data together suggest that Raf-1 is associated with ASK1 in mammalian cells. As a control, FLAG-Raf^{S259/621A} was found to bind HA-ASK1WT (data not shown).

To test whether Raf-1 interacts with ASK1 in the absence of experimental manipulation, we isolated the endogenous Raf-1 protein complex from L929 cells by using an anti-Raf-1 monoclonal antibody and probed for the presence of native ASK1. Indeed, the Raf-1 antibody coimmunoprecipitated endogenous ASK1 (FIG. 3C). As a control, an anti-HA monoclonal antibody failed to pull down ASK1 under the same conditions, suggesting a specific interaction of Raf-1 with ASK1. A reciprocal experiment showed the presence of Raf-1 in immunocomplexes isolated with either anti-ASK1 H300 (Santa Cruz Biotechnology) or anti-ASK1 DAV antibodies (Saitoh et al. (1998) EMBO J. 17:2596-2606 and data not shown). Endogenous ASK1 was also found in complex with Raf-1 in Jurkat T cells (data not shown). Thus, Raf-1 and ASK1 associate in vivo, which supports the model that Raf-1 promotes cell survival in part by antagonizing the ASK1-mediated apoptotic signaling.

Both Raf-1 and ASK1 are capable of binding to 14-3-3 proteins (Morrison and Cutler (1997) Curr. Opin. Cell Biol. 9:174-179, Zhang et al., (1999) Proc. Natl. Acad. Sci. USA 96:8511-8515). The 14-3-3 proteins exist as dimers and could potentially bridge two distinct target proteins (Fu et al. (2000) Annu. Rev. Pharmacol. Toxicol. 40:617-647). It has been demonstrated that 14-3-3 interacts with Raf-1 through phosphorylated Ser-259 and Ser-621 (Morrison and Cutler (1997) Curr. Opin. Cell Biol. 9:174-179) and with ASK1 through phosphorylated Ser-967 (Zhang et al. (1999) Proc. Natl. Acad. Sci. USA 96:8544-8515). Thus, it is possible that the Raf-1/ASK1 association is mediated by 14-3-3 dimers. To test this notion, we used Raf-1 and ASK1 mutants that are defective in 14-3-3 binding, Raf^{S259/621A} and ASK1^{S967A} (Morrison and Cutler (1997) Curr. Opin. Cell Biol. 9:174-179, Zhang et al. (1999) Proc. Natl. Acad. Sci. USA 96:8511-8515). As shown in FIG. 3D, mutant Raf-1 and ASK1 proteins interacted as efficiently as the WT proteins did, suggesting that the Raf-1-ASK1 interaction does not require 14-3-3 proteins.

### Example 4

Raf-1 Catalytic Activity Is Not Required for Inhibition of ASK1-Induced Apoptosis

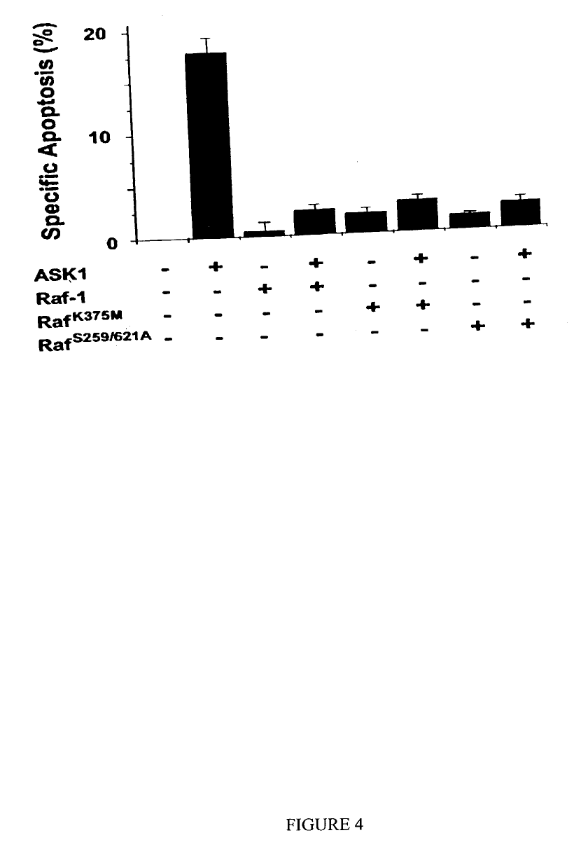

Mutations at Lys-375 and Ser-621 inactivate the kinase activity of Raf-1 (Morrison and Cutler (1997) Curr. Opin. Cell Biol. 9:174-179 and Koich (2000) Biochem. J. 351:289-305). FIG. 4 provides a graphic summary of a HeLa cell morphology-based apoptosis assay performed to determine whether kinase-defective Raf-1 mutants Raf-1^{K375M} and Raf-1^{S259/621A} were capable of binding ASK1. The HeLa cell morphology-based assay described in FIG. 2 was used to score for specific apoptosis. Plasmids used were the same as described in FIG. 3C. The kinase-defective Raf-1 mutants Raf-1^{K375M} and Raf-1^{S259/621A} were capable of binding ASK1 (FIG. 3D). We examined whether the catalytic activity of Raf-1 was required for inhibiting ASK1-induced cell death in HeLa cells. Strikingly, these inactive Raf-1 mutants were as effective as WT in blocking the proapoptotic function of ASK1 under the conditions tested (FIG. 4). Similar results were obtained with COS7 cells in an alternative DNA content-based apoptosis assay using flow cytometry (data not shown). Together, these data suggest a kinase-independent function of Raf-1, strengthening the notion that the MEK-ERK pathway is not involved.

### Example 5

The N-Terminal Regulatory Domain of ASK1 Mediates Raf-1 Binding

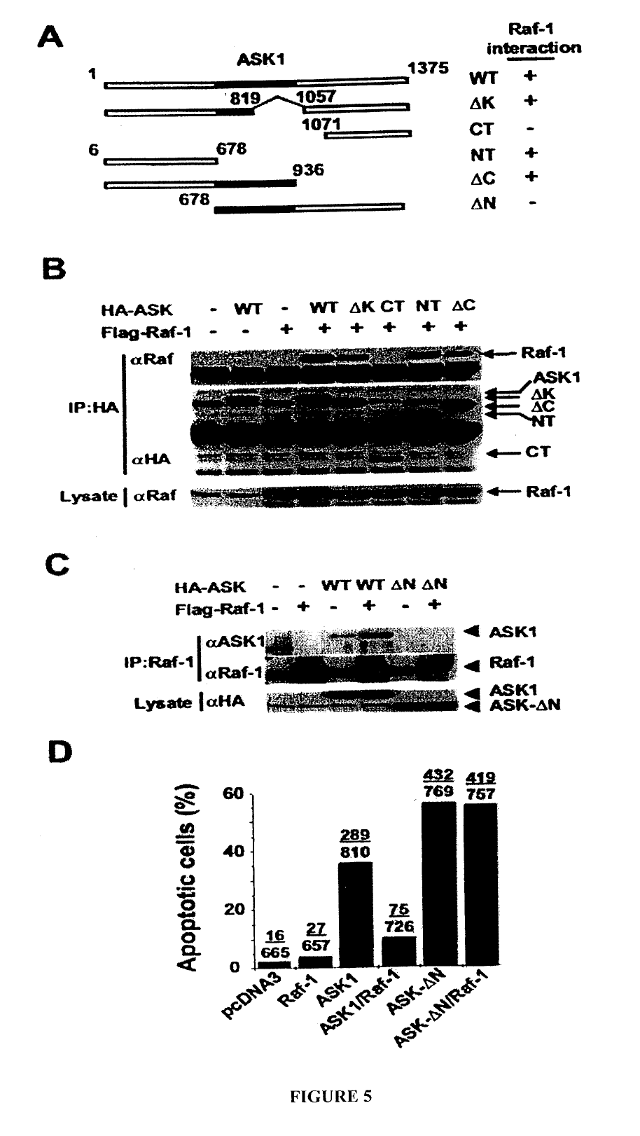

If the Raf-1-ASK1 interaction mediates the inhibitory effect of Raf-1 on ASK1-induced death, we reasoned that a mutant form of ASK1 incapable of Raf-1 binding would be refractory to Raf-1 inhibition. To test this hypothesis, we mapped the Raf-1 binding site on ASK1. ASK1 has its catalytic domain flanked by N-terminal and C-terminal regulatory domains (FIG. 5A). Various truncation mutants of ASK1 were expressed as HA-tagged fusions together with FLAG-Raf-1 in COS7 cells, and their associations were probed in a coimmunoprecipitation assay. FIG. 5B provides the results of a Western blot analysis that established that the N-terminal domain of ASK1 is required for Raf-1 binding. Although all of the ASK1 proteins containing the N-terminal domain showed binding to Raf-1, no Raf-1 binding was detectable for the kinase or C-terminal domains of ASK1 (FIG. 5B and C). Importantly, the N-terminal domain alone was sufficient to bind Raf-1. Raf-1 may inhibit ASK1 by targeting its N-terminal regulatory domain. Deletion analysis localized the Raf-1 binding site of ASK1 to the N-terminal regulatory fragment of the kinase. The invention further provides series of N-terminal truncated ASK1 proteins (e.g., peptides), that are capable of disrupting the Raf1/ASK1 interaction.

The Western blot presented in FIG. 5C demonstrates that Raf-1 does not interact with ASK1-ΔN. Raf-1 protein complexes were immunoprecipitated from each sample with anti-Raf-1 antibody and subjected to SDS/PAGE and Western blotting with anti-ASK1 antibody. Overexposure shows the interaction of endogenous Raf-1 with overexpressed HA-ASK1, but even overexpressed Raf-1 was incapable of binding to ASK1-N.

To test whether binding to ASK1 is necessary for the antiapoptotic activity of Raf-1, we investigated the effect of Raf-1 expression on apoptosis induced by ASK1-ΔN. HeLa cells were transfected with plasmids as indicated together with an eGFP marker vector. In a nuclear morphology-based apoptosis assay, the fraction of apoptotic cells induced by ASK1^{WT} was drastically reduced by coexpression of Raf-1, whereas cell death-induced by ASK1-ΔN was nonresponsive to the coexpressed Raf-1 (FIG. 5D). FIG. 5D is a graphic representation of apoptoisis data obtained in a nuclear morphology-based assay performed to evaluate the effect of the mutations on ASK1-mediated apoptosis as determined by the nuclear morphology-based apoptotic assay described in FIG. 1.

These data strongly support a requirement for the Raf-1-ASK1 interaction in the inhibition of ASK1 proapoptotic function. The results indicate that Raf-1 cannot block ASK1-ΔN induced apoptosis.

### Example 6

ASK1 N-terminal Domain Truncation Peptides

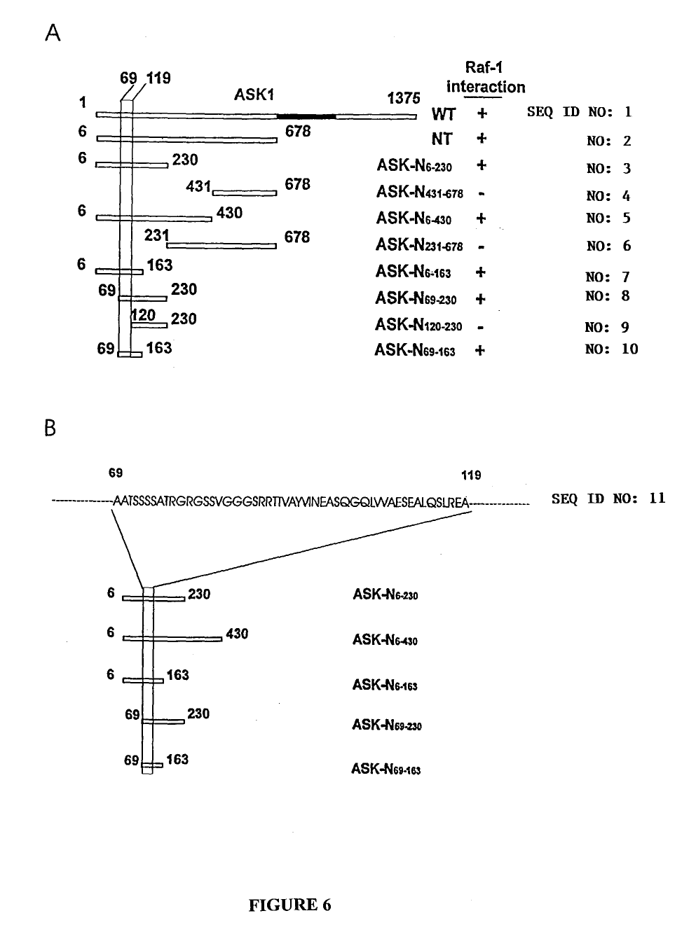

FIG. 6A provides a schematic representation of the ASK1 N-terminal domain truncation peptides used herein. Various truncation mutants of the ASK1 N-terminal domain were generated as shown and expressed as fusion proteins with a (His)₆-tag at the N-terminus and an HA-tag at the C-terminus, except ASK-N₆₉₋₁₆₃ which has the N-terminal (His)6-tag but a GST-tag at the C-terminus. Association of ASK1 mutants with Raf-1 is summarized. Positive interaction is represented by (+).

SEQ ID NO:3 corresponds to amino acid residues 6 to 230 of the full-length human ASK1 sequence set forth in SEQ ID NO:1; SEQ ID NO:5 corresponds to amino acid residues 6 to 430 of the full-length human ASK1 sequence set forth in SEQ ID NO:1; SEQ ID NO:7 corresponds to amino acid residues 6 to 163 of the full-length human ASK1 sequence set forth in SEQ ID NO:1; SEQ ID NO:8 corresponds to amino acid residues 69 to 230 of the full-length human ASK1 sequence set forth in SEQ ID NO:1; SEQ ID NO:10 corresponds to amino acid residues 69 to 163 of the full-length human ASK1 sequence set forth in SEQ ID NO:1 and SEQ ID NO:11 corresponds to amino acid residues 69 to 119 of the full-length human ASK1 sequence set forth in SEQ ID NO:1.

FIG. 6B provides a schematic representation of the ASK1 truncation proteins that inhibit the interaction of ASK1 and Raf-1 which includes the amino acid sequence (SEQ ID NO:11) that is common to all of the ASK1 peptides capable of blocking the Raf-1 interaction.

### Example 7

Expression of ASK1 N-terminal Domain Fusion Peptides Disrupt the Protein-Protein Interaction Between ASK1 and Raf-2

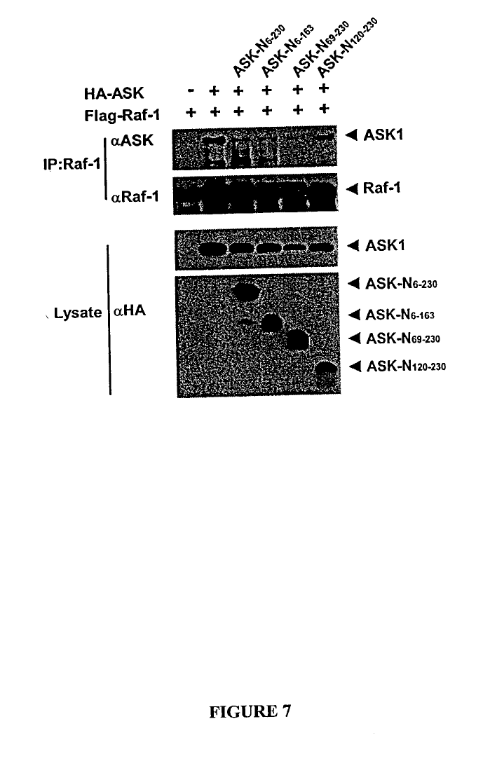

Coimmunoprecipitation experiments were performed using anti-Raf-1 antibody (RI9120, Transduction Laboratories) as described. The Western blot presented in FIG. 7 demonstrates that the expression of Raf-1 binding peptides derived from the N-terminal regulatory domain of ASK1 disrupts the protein-protein interaction between ASK1 and Raf-1. Raf-1 immunoprecipitates were analyzed with antibodies specific for ASK1 and Raf-1 (upper panel). HA-ASK1 protein present in Raf-1 immunoprecipitates was probed with anti-ASK1 antibody H-300 (1:2000, Santa Cruz;). The data presented in the upper panel indicates that the expression of a fusion peptide selected from the group consisting of SEQ ID NOS:3, 7 and 8 disrupts the protein-protein interaction between Raf-1 and ASK1. The effect of this disruption is manifested as the absence of ASK1 protein in the Raf-1 immunoprecipitates. In contrast, expressing a nucleotide sequence encoding a N-terminal domain peptide that does not bind to Raf-1 produces an anti-Raf-1 immunoprecipitate that contains an ASK1 band. The lower panel of FIG. 7 shows the expression levels of each of the N-terminal domain fusion peptides presented in the sample lysates.

### Example 8

Disruption of the Protein-Protein Interaction Between Raf-1/ASK1 Eliminates the Raf-1 Prosurvival Effects on ASK1-induced Cell Death

A DNA content-based flow cytometric apoptosis assay was performed to evaluate the effects of the ASK1 N-terminal domain truncation peptides on the prosurvival effects of Raf-1. COS7 cells were transfected with indicated plasmids encoding HA-ASK1 (1.0 μg), Flag-Raf-1 (0.3 μg) or ASK1 truncation constructs (0.3 μg) as indicated along with an EGFP-F expression vector (0.4 μg) as described. The fraction of transfected cells with sub-G₀ DNA content in each sample was quantified and displayed graphically.

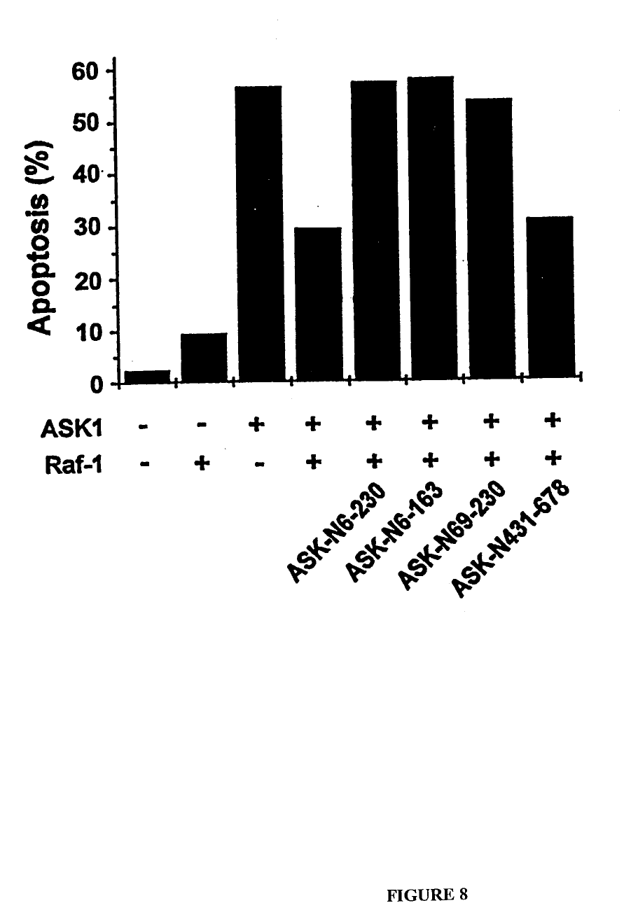

The data presented in FIG. 8 confirms that overexpression of ASK1 promotes apoptosis and that coexpression of Raf-1 antagonizes ASK1-mediated cell death. The data presented in FIG. 8 is representative of three independent experiments. The data indicates that expression of a nucleotide sequence encoding a fusion peptide characterized by an ability to bind Raf-1, such as SEQ ID NOS:3, 7 and 8 disrupts the Raf-1/ASK1 association and eliminates Raf-1 prosurvival effects on ASK1-induced cell death. Expression of a nucleotide sequence encoding a N terminal domain fusion peptide that does not bind Raf-1 (such as the peptide encoded by the amino acid sequence provided in SEQ ID NO:4) does not antagonize the prosurvival effect of Raf-1 expression.

Discussion

The data presented herein suggest a mechanism by which Raf-1 promotes cell survival independently of the MEK-ERK pathway. Through protein-protein interactions, Raf-1 may directly act on a critical component of the cellular proapoptotic signaling machinery. The fact that catalytically inactive Raf-1 can replace the WT kinase to inhibit ASK1 raises the intriguing possibility that Raf-1 may have a kinase-independent function. It is conceivable that the interaction of Raf-1 with many reported targets may represent kinase-independent pathways of Raf-1 (Kolch, W. (2000) Biochem. J. 351:289-305). Thus, Raf-1 may have dual functions of activating the MEK-ERK cascade through its enzymatic activity while inhibiting ASK1 through protein-protein interactions.

Evidence is accumulating that Raf-1 may use multiple effectors, in addition to its well established target MEK, to mediate its cellular functions. It was found that activated Raf-1, but not MEK, can drive the differentiation of hippocampal neuronal cells (Kuo et al. (1996) Mol. Cell. Biol. 16:1458-1470). Mutant Raf-1 that is defective in MEK activation is still capable of activating NF-B-dependent gene expression and other selected pathways (Pearson et al. (2000) J. Biol. Chem. 275:37303-37306) remain to be identified. However, these observations together strongly support the notion that Raf-1 can transmit signals to multiple downstream pathways.

Consistent with this idea, Raf-1 has been shown to interact with other critical regulatory proteins such as the cell-cycle modulators Cdc25 and Rb (Galaktionov et al. (1995) Genes Dev. 9:1046-1058 and Wang et al. (1998) Mol. Cell. Biol. 18:7487-7498) and the proapoptotic protein Bad (Wang et al. (1996) Cell 87:629-638). ASK1 was initially described as a MAPK kinase kinase that activates the stress-activated protein kinases SAPK/JNK and p38 (Ichijo et al. (1997) Science 275:90-94 and Wang et al. (1996) J. Biol. Chem. 271:31607-31611). Interaction of Raf-1 with ASK1 may allow functional cross-talk between two antagonistic signaling pathways, which is likely to be critical for signal integration. Recent demonstration of the interaction between Raf-1 and MEKK1 supports an extensive interplay at the MAPK kinase kinase level of the signaling network (Karandikar et al. (2000) J. Biol. Chem. 275:40120-40127). The concerted action of Raf-1 on several aspects of cell growth control may prevent conflicting signaling activities and lead to a meaningful biological output.

It has been postulated that apoptotic cell death is the default program of metazoan cells which must be suppressed continuously by survival mechanisms (Jacobson et al. (1997) Cell 88:347-354). Inhibition of the proapoptotic function of ASK1 by Raf-1 may be part of the cellular machinery that maintains survival. It is conceivable that activation of ASK1-mediated apoptosis by death stimuli such as H₂O₂ and tumor necrosis factor α may involve the dissociation of Raf-1 from ASK1. Because Raf-1 is a vital component of a variety of growth factor-induced signaling pathways, simultaneous stimulation of the MEK-ERK pathway and inhibition of death signaling through its kinase-dependent and independent mechanisms may both be necessary to ensure cell survival and proliferation. How Raf-1 inhibits ASK1 remains to be established. It is possible that Raf-1 promotes an inactive conformation of ASK1 through the N-terminal domain of ASK1, removal of which has been shown to increase both the kinase activity of ASK1 and its lethality (Chang et al. (1998) Science 281:1860-1863). It is also possible that Raf-1 binding interferes with the interaction of ASK1 with its effectors such as MKK3 or regulators such as Daxx (Ichijo et al. (1997) Science 275:90-94 and Chang et al. (1998) Science 281:1860-1863).

Alternatively, it is tempting to speculate that Raf-1 may function as an adaptor protein to recruit a survival factor to inhibit ASK1 function. For example, Raf-1 interacts with Akt (Zimmermann and Moelling (1999) Science 286:1741-1744 and Rommel et al. (1999) Science 286:1738-1741), a phosphoinositide 3-kinase regulated prosurvival kinase. Thus Raf-1 may recruit Akt to phosphorylate ASK1, allowing a general survival mechanism to intercept a death-signaling pathway (Kim et al. (2001) Mol. Cell. Biol. 21:893-901).

Together, the data of the invention show that Raf-1 interacts with ASK1, and this interaction allows Raf-1 to inhibit a critical mediator of cell death independently of the MEK-ERK pathway, possibly through a kinase-independent mechanism. Investigations into the physiological roles of Raf-1 must now consider not only its MEK kinase activity but also Raf-1-mediated protein-protein interactions.

All publications and patent applications mentioned in the specification are indicative of the level of those skilled in the art to which this invention pertains. All publications and patent applications are herein incorporated by reference, to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

What is claimed is:

1. A method of screening for agents that increase or decrease the binding of ASK1, or an N-terminal fragment thereof, to a Raf-1 binding target, comprising:
a) contacting ASK1, or an N-terminal fragment thereof, and a Raf-1 binding target in the presence of an agent;
b) measuring the binding of said ASK1, or said N-terminal fragment thereof, to said Raf-1 binding target; and
c) determining whether said binding has been increased or decreased.

2. The method of claim 1, wherein said Raf-1 binding target is selected from the group consisting of Raf-1, a catalytically inactive Raf-1, a fragment of Raf-1, and a fragment of a catalytically inactive Raf-1.

3. The method of claim 1, wherein said N-terminal fragment of ASK1 comprises the amino acid sequence of SEQ ID NO:11.

4. The method of claim 1, wherein said N-terminal fragment of ASK1 is selected from the group consisting of SEQ ID NOS:3, 5, 7, 8, 10, and 11.

5. The method of claim 1, wherein said agent is selected from the group consisting of:
a) an antibody that specifically binds to a polypeptide selected from the group consisting of SEQ ID NOS:3, 5, 7, 8, 10, and 11;
b) a peptidomimetic which is structurally similar to a paradigm polypeptide selected from the group consisting of SEQ ID NOS:3, 5, 7, 8, 10, and 11;
c) a polypeptide comprising the amino acid sequence of SEQ ID NO:11;
d) a fragment of SEQ ID NO:3; and
e) a fragment of Raf-1.

6. The method of claim 1, wherein said ASK1, or said N-terminal fragment thereof, and said Raf-1 binding target are contacted in vitro.

7. The method of claim 1, wherein said ASK1, or said N-terminal fragment thereof, and said Raf-1 binding target are contacted in vivo.

8. The method of claim 1, wherein said binding is determined using a method selected from the group consisting of a coimmunoprecipitation assay, a fluorescent polarization assay, and a two-hybrid assay.

9. The method of claim 1, wherein said binding is determined by measuring the level of ASK1-induced apopotosis in the presence of said agent versus the level of ASK1-induced apopotosis in the absence of said agent, and wherein ASK1 is the full-length ASK1 polypeptide.

10. A method of determining whether a Raf-1 binding target is bound by ASK1, comprising:
a) contacting a Raf-1 binding target and ASK1, or an N-terminal fragment thereof, and
b) measuring the binding of said Raf-1 binding target and ASK1, or an N-terminal fragment thereof.

11. The method of claim 10, wherein said Raf-1 binding target is selected from the group consisting of:
a) a fragment of Raf-1;
b) a fragment of a catalytically inactive Raf-1;
c) a Raf-1 having a deletion mutation; and
d) a catalytically inactive Raf-1 having a deletion mutation.

12. The method of claim 10, wherein said binding is determined using a method selected from the group consisting of a coimmunoprecipitation assay, a fluorescent polarization assay, and a two-hybrid assay.

13. The method of claim 10, wherein said binding is determined by measuring the level of ASK1-induced apopotosis in the presence of said Raf-1 binding target versus the level of ASK1-induced apopotosis in the absence of said Raf-1 binding target, and wherein ASK1 is the full-length ASK1 polypeptide.

14. A method of increasing or decreasing the regulation by Raf-1 of ASK1 induced apoptosis, comprising administering an agent that increases or decreases the interaction between Raf-1 and ASK1.

15. The method of claim 14, wherein said agent is selected from the group consisting of:
a) an antibody that specifically binds to a polypeptide selected from the group consisting of SEQ ID NOS:3, 5, 7, 8, 10, and 11;
b) a peptidomimetic which is structurally similar to a paradigm polypeptide selected from the group consisting of SEQ ID NOS:3, 5, 7, 8, 10, and 11;
c) a polypeptide comprising the amino acid sequence of SEQ ID NO:11;
d) a fragment of SEQ ID NO:3; and
e) a fragment of Raf-1.

16. A method of decreasing ASK1-induced apoptosis, comprising administering an agent that decreases the ability of ASK1 to induce apoptosis by binding to the N-terminal region of ASK1.

17. The method of claim 16, wherein said agent is selected from the group consisting of:
a) an antibody that specifically binds to a polypeptide selected from the group consisting of SEQ ID NOS:3, 5, 7, 8, 10, and 11; and
b) a fragment of Raf-1.

18. An antibody that specifically binds to an amino acid sequence selected from the group consisting of SEQ ID NOS:1, 3, 5, 7, 8, 10, and 11.

19. An isolated polypeptide or peptidomimetic that is bound by an antibody of claim 18.

20. A method of inhibiting ASK1-induced apoptosis in a cell comprising introducing into said cell a nucleic acid encoding a catalytically inactive Raf-1 binding target capable of specifically binding the N-terminal regulatory domain of ASK1, and expressing said nucleic acid in said cell such that the resultant gene product inhibits apoptosis in said cell.

21. A method of inhibiting TNF α-mediated apoptosis in a cell comprising introducing into said cell a nucleic acid encoding a catalytically inactive Raf-1 binding target capable of specifically binding the N-terminal regulatory domain of ASK1, and expressing said nucleic acid in said cell such that the resultant gene product inhibits TNF α-mediated apoptosis in said cell.
